(19) United States
(12) Patent Application Publication
(10) Pub. No.: US 2024/0350629 A1
(43) Pub. Date: Oct. 24, 2024

(54) GENETICALLY ENGINEERED CELL-DERIVED VACCINES

(71) Applicant: THE REGENTS OF THE UNIVERSITY OF CALIFORNIA, Oakland, CA (US)

(72) Inventors: Young Jik Kwon, Irvine, CA (US); Jee Young Chung, Irvine, CA (US)

(21) Appl. No.: 18/681,457

(22) PCT Filed: Aug. 12, 2022
(86) PCT No.: PCT/US2022/040243

§ 371 Date: Feb. 5, 2024

Related U.S. Application Data
(60) Provisional application No. 63/233,190, filed on Aug. 13, 2021.

(51) Int. Cl. A61K 39/00; A61K 39/215; A61P 37/04

(52) U.S. Cl. CPC A61K 39/4622; A61K 39/215; A61K 39/4612; A61K 39/4614; A61K 39/4615; A61K 39/4641; A61K 39/4644; A61K 39/4648; A61K 39/464838; A61P 37/04

(57) ABSTRACT

The disclosure provides for compositions and methods comprising cell-derived vesicles induced from cells that have been genetically engineered or infected to express specific antigen(s), and uses thereof, including as a cell-free, cell-like vaccine.

### CROSS REFERENCE TO RELATED APPLICATIONS

This application claims priority under 35 U.S.C. § 119 from Provisional Application Ser. No. 63/233,190 filed Aug. 13, 2021. the disclosure of which is incorporated herein by reference.

### TECHNICAL FIELD

The disclosure provides for compositions and methods comprising cell-derived vesicles induced from cells that have been genetically engineered or infected to express specific antigen(s), and uses thereof, including as a cell-free, cell-like vaccine.

### BACKGROUND

The use of vaccines against infectious diseases has been critical to the advancement of medicine. Coronaviruses (CoVs) are a group of related viruses that can cause respiratory infections in humans ranging from mild symptoms and lethal outcomes. Human Coronavirus 229E (HCoV-229E), OC43 (HCoV-OC43), NL63 (HCoV-NL63), and HKU1 (HCoV-HKU1), are known to cause relatively mild and self-limiting respiratory symptoms. Alternatively, the other three CoVs, Severe Acute Respiratory Syndrome Coronavirus (SARS-CoV), Middle East Respiratory Syndrome Coronavirus (MERS-CoV), and Severe Acute Respiratory Syndrome Coronavirus 2 (SARS-CoV-2), are highly pathogenic and can lead to severe respiratory diseases and fatal outcome in infected patients. The clinical manifestation of these three coronaviruses can vary from asymptomatic and mild flu-like symptoms to acute respiratory distress syndrome and death. Compared to SARS-CoV and MERS-CoV, SARS-CoV-2 is highly contagious and effective vaccines are needed to mitigate the pandemic. Multiple SARS-CoV-2 vaccines, developed by different manufactures, have been approved by the Federal Drug Administration.

The high morbidity and mortality rate of COVID-19 has necessitated the rapid development of vaccines against severe acute respiratory syndrome coronavirus (SARS-CoV-2). Vaccines based on mRNA, viral vectors, and recombinant protein have been rapidly developed and have shown effective protection against the earliest strains of SARS-CoV-2. These vaccines, however, exhibit mild and even severe side effects, and the protection they elicit is of short duration. The vaccines are notably less effective against emerging strains of SARS-CoV-2, such as the Omicron strain.

Platform technologies have been employed to develop vaccine candidates, such as nucleic acid platforms, non-replicating viral vectored platforms, inactivated virus, or recombinant subunit vaccines. These vaccines employ administration of viral antigens or viral gene sequences to induce neutralizing antibodies against the viral spike(S) protein. Conventional vaccines involve entire organisms or large proteins, which leads to unnecessary antigenic load along with increased chances of allergenic responses. The drawbacks of such vaccines can be overcome by genetically engineering specific antigen-based vaccines compromising short immunogenic fragments with the ability to elicit strong and targeted immune responses, avoiding the chances of allergenic reactions. Genetically engineered vaccines can induce the expression of target antigenic epitopes in the body of an immunized subject, and elicit an immune response, including humoral and cellular immune responses.

### SUMMARY

The disclosure provides preparations (e.g., vaccine preparations), compositions (e.g., pharmaceutical compositions) and methods comprising cell-derived vesicles induced from antigen presenting cells (e.g., dendritic cells) or non-antigen presenting cells that have been engineered to express an antigen (e.g., a foreign antigen). In the studies presented herein, it was shown that extracellular blebs produced from engineered dendritic cells expressing SARS-CoV-2 spike glycoprotein elicited a strong immune response in vivo. The disclosure further provides methods to genetically engineer cells, to induce blebbing in said cells with a blebbing agent. isolation of the extracellular blebs (EBs) produced thereof, and preparations (e.g., vaccine preparations), compositions (e.g., pharmaceutical compositions) comprising the EBs. Additionally, cells infected with viruses, bacteria, or fungi can be also be used in the methods of the disclosure for vaccine preparations (e.g., SARS-CoV-2 infected lung epithelium cells to be blebbed for COVID-19 vaccines. Chlamydia-infected cervical cells for Chlamydia vaccines, and more).

In a particular embodiment, the disclosure provides for a vaccine preparation comprising induced cell-derived vesicles from a cell that has been genetically engineered to express an antigen(s), wherein the EBS are generated from the cell by treating the antigen cell with a blebbing agent. In another embodiment, the cell is selected from a macrophage, a B cell and a dendritic cell. In yet another embodiment, the cell is a dendritic cell. In a further embodiment, the cell is an immortalized antigen presenting cell line. In yet a further embodiment, the cell is differentiated from human embryonic stem cells (hESCs) or induced pluripotent stem cells (iPSCs) from a human subject. In another embodiment, the cell is a human primary cell. In yet another embodiment, the antigen(s) is a foreign antigen(s) or an endogenous antigen that has been genetically modified for improved therapeutic outcomes. In a further embodiment, the foreign antigen(s) is from a pathogenic or disease-causing microorganism. In yet a further embodiment, the pathogenic or disease-causing microorganism is a bacterium, a fungus, or a virus. In a certain embodiment, the bacterium is selected from the group consisting of Actinomyces israelii, Bacillus anthracis, Bacillus cereus, Bartonella henselae, Bartonella quintana, Bordetella pertussis, Borrelia burgdorferi, Borrelia garinii, Borrelia afzelii, Borrelia recurrentis, Brucella abortus. Brucella canis. Brucella melitensis, Brucella suis, Campylobacter jejuni, Chlamydia pneumoniae, Chlamydia trachomatis, Chlamydophila psittaci, Clostridium botulinum, Clostridium difficile, Clostridium perfringens, Clostridium tetani, Corynebacterium diphtheriae, Enterococcus faecalis, Enterococcus faecium, Escherichia coli, Francisella tularensis, Haemophilus influenzae. Helicobacter pylori, Legionella pneumophila. Leptospira interrogans, Leptospira santarosai, Leptospira weilii, Leptospira noguchii, Listeria monocytogenes, Mycobacterium leprae, Mycobacterium tuberculosis, Mycobacterium ulcerans. Mycoplasma pneumoniae, Neisseria gonorrhoeae. Neisseria meningitidis, Pseudomonas aeruginosa. Rickettsia rickettsia, Salmonella typhi, Salmonella typhimurium, Shigella sonnei, Staphylococcus aureus, Staphylococcus epidermidis, Staphylococcus saprophyticus, Streptococcus agalactiae, Streptococcus pneumoniae, Streptococcus pyogenes, Treponema pallidum, Ureaplasma urealyticum, Vibrio cholerae, Yersinia pestis, Yersinia enterocolitica, and Yersinia pseudotuberculosis. In another embodiment, the fungus is selected from the group consisting of Absidia corymbifera, Absidia ramose, Achorion gallinae, Actinomadura spp., Ajellomyces dermatididis, Aleurisma brasiliensis, Allersheria boydit, Arthroderma spp., Aspergillus flavus, Aspergillus fumigatu, Basidiobolus spp, Blastomyces spp, Cadophora spp., Candida albicans, Cercospora apii, Chrysosporium spp, Cladosporium spp, Cladothrix asteroids, Coccidioides immitis, Cryptococcus albidus, Cryptococcus gattii, Cryptococcus laurentii, Cryptococcus neoformans, Cunninghamella elegans, Dematium wernecke, Discomyces israelii, Emmonsia spp, Emmonsiella capsulate, Endomyces geotrichum, Entomophthora coronate, Epidermophyton floccosum, Filobasidiella neoformans, Fonsecaea spp., Geotrichum candidum, Glenospora khartoumensis, Gymnoascus gypseus, Haplosporangium parvum, Histoplasma, Histoplasma capsulatum, Hormiscium dermatididis, Hormodendrum spp., Keratinomyces spp, Langeronia soudanense, Leptosphaeria senegalensis, Lichtheimia corymbifera, Lobmyces loboi., Loboa loboi, Lobomycosis, Madurella spp., Malassezia furfur, Micrococcus pelletieri, Microsporum spp, Monilia spp., Mucor spp., Mycobacterium tuberculosis, Nannizzia spp., Neotestudina rosatii, Nocardia spp., Oidium albicans, Oospora lactis, Paracoccidioides brasiliensis, Petriellidium boydii, Phialophora spp., Piedraia hortac, Pityrosporum furfur, Pneumocystis jirovecii (or Pneumocystis carinii), Pullularia gougerotti, Pyrenochaeta romeroi, Rhinosporidium seeberi, Sabouraudites (Microsporum), Sartorya fumigate, Sepedonium, Sporotrichum spp., Stachybotrys, Stachybotrys chartarum, Streptomyce spp., Tinea spp., Torula spp, Trichophyton spp, Trichosporon spp, and Zopfia rosatii. In yet another embodiment, the virus is selected from the group consisting of Adeno-associated virus, Aichi virus, Australian bat lyssavirus, BK polyomavirus, Banna virus, Barmah forest virus, Bunyamwera virus, Bunyavirus La Crosse, Bunyavirus snowshoe hare, Cercopithecine herpesvirus, Chandipura virus, Chikungunya virus, Cosavirus A, Coronavirus, Cowpox virus, Coxsackievirus, Crimean-Congo hemorrhagic fever virus, Dengue virus, Dhori virus, Dugbe virus, Duvenhage virus, Eastern equine encephalitis virus, Ebolavirus, Echovirus, Encephalomyocarditis virus. Epstein-Barr virus. European bat lyssavirusalitis, GB virus C/Hepatitis G virus Pegivirus, Hantan virus, Hendra virus, Hepatitis A virus, Hepatitis B virus, Hepatitis C virus, Hepatitis E virus, Hepatitis delta virus, Horsepox virus, Human adenovirus, Human astrovirus, Human coronavirus, Human cytomegalovirus, Human enterovirus, Human herpesvirus, Human immunodeficiency virus, Human papillomavirus, Human parainfluenza, Human parvovirus B19, Human respiratory syncytial virus, Human rhinovirus, Human SARS coronavirus, Human spumaretrovirus, Human T-lymphotropic virus, Human torovirus, Influenza A virus, Influenza B virus, Isfahan virus, JC polyomavirus, Japanese encephalitis virus, Junin arenavirus, KI Polyomavirus, Kunjin virus, Lagos bat virus, Lake Victoria Marburgvirus, Langat virus, Lassa virus, Lordsdale virus, Louping ill virus, Lymphocytic choriomeningitis virus, Machupo virus, Mayaro virus, MERS coronavirus, Measles virus, Mengo encephalomyocarditis virus, Merkel cell polyomavirus, Mokola virus, Molluscum contagiosum virus, Monkeypox virus, Mumps virus, Murray valley encephalitis virus, New York virus, Nipah virus, Norwalk virus, O'nyong-nyong virus, Orf virus, Oropouche virus, Pichinde virus, Poliovirus, Punta toro phlebovirus, Puumala virus, Rabies virus, Rift valley fever virus, Rosavirus A, Ross river virus, Rotavirus A, Rotavirus B, Rotavirus C, Rubella virus, Sagiyama virus, Salivirus A, Sandfly fever sicilian virus, Sapporo virus, Semliki forest virus, Seoul virus, Simian foamy virus, Simian virus, Sindbis virus, Southampton virus, St. louis encephalitis virus, Tick-borne powassan virus, Torque teno virus, Toscana virus, Uukuniemi virus, Vaccinia virus, Varicella-zoster virus, Variola virus O, Venezuelan equine encephalitis virus, Vesicular stomatitis virus, Western equine encephalitis virus, WU polyomavirus, West Nile virus, Yaba monkey tumor virus, Yaba-like disease virus, Yellow fever virus, and Zika virus. In a particular embodiment, the human coronavirus is SARS-CoV-2. In a further embodiment, the antigen presenting cell is engineered by introducing a viral vector. In yet a further embodiment, the viral vector is a lentivirus vector, an adenovirus vector, an adeno-associated virus vector, or a gammaretrovirus vector. In another embodiment, the viral vector is a lentivirus vector. In yet another embodiment, the vaccine preparation does not require an adjuvant but an adjuvant could be included.

In a certain embodiment, the disclosure also provides a method of making a vaccine preparation of the disclosure, comprising: inducing cell-derived vesicles from a genetically engineered cell by contacting the cell with the one or more sulfhydryl blocking agents for 3 min to 24 h; and isolating the cell-derived vesicles. In a further embodiment, the one or more sulfhydryl blocking agents are selected from the group consisting of mercury chloride, p-chloromercuribenzene sulfonic acid, auric chloride, p-chloromercuribenzoate, chlormerodrin, meralluride sodium, iodoacetmide, paraformaldehyde, dithiothreitol, and N-ethylmaleimide. In yet a further embodiment, the one or more sulfhydryl blocking agents is N-ethylmaleimide. In yet a further embodiment, N-ethylmaleimide is used at a concentration of 0.2 mM to 30 mM. In another embodiment, the cell is selected from a macrophage, a B cell and a dendritic cell. In yet another embodiment, the cell is a dendritic cell. In a further embodiment, the cell is an immortalized antigen presenting cell. In yet a further embodiment, the cell is differentiated from human embryonic stem cells (hESCs) or induced pluripotent stem cells (iPSCs) from a human subject. In another embodiment, the cell is a human primary cell. In yet another embodiment, the antigen(s) is a foreign antigen(s) or an endogenous antigen that has been genetically modified for improved therapeutic outcomes. In a particular embodiment, the foreign antigen(s) is from a pathogenic or disease-causing microorganism. In another embodiment. the pathogenic or disease-causing microorganism is a bacterium, a fungus, or a virus. In yet another embodiment, the bacterium is selected from Actinomyces israelii, Bacillus anthracis, Bacillus cereus, Bartonella henselae, Bartonella quintana, Bordetella pertussis, Borrelia burgdorferi, Borrelia garinii, Borrelia afzelii, Borrelia recurrentis, Brucella abortus, Brucella canis, Brucella melitensis, Brucella suis, Campylobacter jejuni, Chlamydia pneumoniae, Chlamydia trachomatis, Chlamydophila psittaci, Clostridium botulinum, Clostridium difficile. Clostridium perfringens, Clostridium tetani, Corynebacterium diphtheriae, Enterococcus faecalis, Enterococcus faecium, Escherichia coli, Francisella tularensis, Haemophilus influenzae, Helicobacter pylori, Legionella pneumophila, Leptospira interrogans, Leptospira santarosai, Leptospira weilii, Leptospira noguchii, Listeria monocytogenes, Mycobacterium leprae, Mycobacterium tuberculosis, Mycobacterium ulcerans, Mycoplasma pneumoniae, Neisseria gonorrhoeae. Neisseria meningitidis, Pseudomonas aeruginosa, Rickettsia rickettsia, Salmonella typhi, Salmonella typhimurium, Shigella sonnet, Staphylococcus aureus, Staphylococcus epidermidis, Staphylococcus saprophyticus, Streptococcus agalactiae, Streptococcus pneumoniae, Streptococcus pyogenes, Treponema pallidum, Ureaplasma urealyticum, Vibrio cholerae, Yersinia pestis, Yersinia enterocolitica, and Yersinia pseudotuberculosis. In a further embodiment, the fungus is selected from Absidia corymbifera, Absidia ramose, Achorion gallinae, Actinomadura spp., Ajellomyces dermatididis, Aleurisma brasiliensis, Allersheria boydii, Arthroderma spp., Aspergillus flavus, Aspergillus fumigatu, Basidiobolus spp, Blastomyces spp, Cadophora spp, Candida albicans, Cercospora apii, Chrysosporium spp, Cladosporium spp, Cladothrix asteroids, Coccidioides immitis, Cryptococcus albidus, Cryptococcus gattii, Cryptococcus laurentii, Cryptococcus neoformans, Cunninghamella elegans, Dematium wernecke, Discomyces israelii, Emmonsia spp, Emmonsiella capsulate, Endomyces geotrichum, Entomophthora coronate, Epidermophyton floccosum, Filobasidiella neoformans, Fonsecaea spp., Geotrichum candidum, Glenospora khartoumensis, Gymnoascus gypseus, Haplosporangium parvum, Histoplasma, Histoplasma capsulatum, Hormiscium dermatididis, Hormodendrum spp., Keratinomyces spp. Langeronia soudanense, Leptosphaeria senegalensis, Lichtheimia corymbifera, Lobmyces loboi., Loboa loboi, Lobomycosis, Madurella spp., Malassezia furfur, Micrococcus pelletieri, Microsporum spp, Monilia spp., Mucor spp., Mycobacterium tuberculosis, Nannizzia spp., Neotestudina rosatii, Nocardia spp., Oidium albicans, Oospora lactis, Paracoccidioides brasiliensis, Petriellidium boydii, Phialophora spp., Piedraia hortae, Pityrosporum furfur, Pneumocystis jirovecii (or Pneumocystis carinii), Pullularia gougerotii, Pyrenochaeta romeroi, Rhinosporidium seebert, Sabouraudites (Microsporum), Sartorya fumigate, Sepedonium. Sporotrichum spp., Stachybotrys, Stachybotrys chartarum, Streptomyce spp., Tinea spp., Torula spp, Trichophyton spp, Trichosporon spp, and Zopfia rosatii. In yet a further embodiment, the virus is selected from the group consisting of Adeno-associated virus, Aichi virus, Australian bat lyssavirus, BK polyomavirus, Banna virus, Barmah forest virus, Bunyamwera virus, Bunyavirus La Crosse, Bunyavirus snowshoe hare, Cercopithecine herpesvirus, Chandipura virus, Chikungunya virus, Cosavirus A, Coronavirus, Cowpox virus, Coxsackievirus, Crimean-Congo hemorrhagic fever virus, Dengue virus, Dhori virus. Dugbe virus, Duvenhage virus, Eastern equine encephalitis virus, Ebolavirus, Echovirus, Encephalomyocarditis virus, Epstein-Barr virus, European bat lyssavirusalitis, GB virus C/Hepatitis G virus Pegivirus, Hantan virus, Hendra virus, Hepatitis A virus, Hepatitis B virus, Hepatitis C virus, Hepatitis E virus, Hepatitis delta virus, Horsepox virus, Human adenovirus, Human astrovirus, Human coronavirus, Human cytomegalovirus, Human enterovirus, Human herpesvirus, Human immunodeficiency virus, Human papillomavirus, Human parainfluenza, Human parvovirus B19, Human respiratory syncytial virus, Human rhinovirus, Human SARS coronavirus, Human spumaretrovirus, Human T-lymphotropic virus, Human torovirus, Influenza A virus, Influenza B virus, Isfahan virus, JC polyomavirus, Japanese encephalitis virus, Junin arenavirus, KI Polyomavirus, Kunjin virus, Lagos bat virus, Lake Victoria Marburgvirus, Langat virus, Lassa virus, Lordsdale virus, Louping ill virus, Lymphocytic choriomeningitis virus, Machupo virus, Mayaro virus, MERS coronavirus, Measles virus, Mengo encephalomyocarditis virus, Merkel cell polyomavirus, Mokola virus, Molluscum contagiosum virus, Monkeypox virus, Mumps virus, Murray valley encephalitis virus, New York virus, Nipah virus, Norwalk virus, O'nyong-nyong virus, Orf virus, Oropouche virus, Pichinde virus, Poliovirus, Punta toro phlebovirus, Puumala virus, Rabies virus, Rift valley fever virus, Rosavirus A, Ross river virus, Rotavirus A, Rotavirus B, Rotavirus C, Rubella virus, Sagiyama virus, Salivirus A, Sandfly fever sicilian virus, Sapporo virus, Semliki forest virus, Seoul virus, Simian foamy virus, Simian virus, Sindbis virus, Southampton virus, St. louis encephalitis virus, Tick-borne powassan virus, Torque teno virus, Toscana virus, Uukuniemi virus, Vaccinia virus. Varicella-zoster virus, Variola virus O, Venezuelan equine encephalitis virus, Vesicular stomatitis virus, Western equine encephalitis virus, WU polyomavirus, West Nile virus, Yaba monkey tumor virus, Yaba-like disease virus, Yellow fever virus, and Zika virus. In a certain embodiment, the human coronavirus is SARS-CoV-2. In another embodiment, the antigen is a cancer or tumor antigen. In yet another embodiment, the genetically engineered cells are made by transforming the cells with a viral vector that encodes the antigen(s). In yet another embodiment, the viral vector is a lentivirus vector, an adenovirus vector, an adeno-associated virus vector, or a gammaretrovirus vector. In a certain embodiment, the viral vector is a lentivirus vector.

In a particular embodiment, the disclosure further provides a method of immunizing a subject, comprising administering a therapeutically effective amount of a vaccine preparation disclosed herein.

### DESCRIPTION OF DRAWINGS

FIG. 1A-C presents the preparation and characterization of EBs derived from SARS-CoV-2 spike protein(S)-expressing DC2 4 cells. (A) Lentiviral transduction of DC2.4 cells for S expression, followed blebbing and vaccination of the syngeneic mice. Briefly, DC2.4 cells were transduced with S-expressing lentivirus prior to puromycin selection. DC2.4 S cells were then treated with blebbing buffer to produce EBs. The micro-sized EBs were isolated by centrifugation and used to vaccinate the animals, followed by assays for antibody binding to S and neutralization of S-pseudotyped virus. (B) DC2.4 cells, DC2.4 S cells, DC2.4 EBs and DC2.4 S EBs were analyzed for S expression by flow cytometry. (C) 2.5×10⁵ DC2.4 S cells and DC2 4 S EBs at a surface area equivalent to 2.5×10⁵ DC2.4 S cells were lysed and their S contents were quantified by ELISA.

FIG. 2 presents images of extracellular blebs (EBs) induced from DC 2.4 cells and DC 2.4 spike cells using 2 mM N-ethylmaleimide (NEM.)

FIG. 3 presents the surface area of DC2.4 and DC2.4 S cells and their extracellular blebs (EBs). The cells were labeled with PKH26 before blebbing. After isolation and purification, EBs produced from 2.5×10⁵ DC2.4 and DC2.4 S cells were lysed and quantified by the fluorescence of PKH26 at 570 nm Em/590 nm Ex. The EBs showed similar fluorescence intensity to their parent cells, indicating highly preserved membrane conversion.

FIG. 4 provides surface marker analyses of DC2.4 S cells and DC2.4 S EBs. DC2.4 cells were transduced using a SARS-CoV-2 spike protein(S)-expressing lentivirus and used for the preparation of DC2.4 S EBs. Both DC2.4 S cells and DC2.4 S EBs were labeled for CD11c, MHC I, CD40, CD80, and CD86 (Biolegend, CA, USA) by staining with fluorescently labeled antibodies before analysis by flow cytometry. Notably, the expression of CD11c, CD80, and CD86 was slightly higher on DC2.4 S EBs than on the DC2.4 S cells.

FIG. 5 presents DC 2.4 cells analyzed for co-stimulatory molecule expression using flow cytometry. DC 2.4 cells were labeled with a fluorescence antibody against CD11c, CD40, CD80, CD86, and MHC I (Biolegend, CA, USA) before and after LPS activation.

FIG. 6 shows DC 2.4 spike cells analyzed for co-stimulatory molecule expression using flow cytometry. Dendritic cells were labeled with fluorescence antibodies against CD11c, CD40, CD80, CD86, and MHC I (Biolegend, CA, USA) before and after LPS activation.

FIG. 7 provides a schematic illustration for in vivo vaccination experiments.

FIG. 8 presents the results of spike IgG ELISAs from mice sera. Sera was collected from mice on Day 0 (Prime), Day 14 (Booster) and Day 24 (10 days after booster) after immunization with PBS, spike protein (10 μg/mouse), equivalent spike protein of 2.5×10⁵ cells (15 ng/mouse), 2.5×10⁵ DC 2.4 cell and DC 2.4-spike cell, equivalent amount by surface area of 2.5×10⁵ DC 2.4 and DC 2.4 spike EBs.

FIG. 9 presents the results of a neutralization assay obtained from mice sera Sera and pseudotyped SARS-CoV-2 spike GFP lentivirus were incubated for 1 hour at 37° C. After incubation, 1×10⁴ ACE-2 expressing 293T cells were added and incubated for 48 hours and analyzed using flow cytometry. Mean fluorescence intensity (MFI) was used to determine the IC₅₀ of the sera sample

FIG. 10 shows the results of a spike IgG ELISA from mouse sera. Sera was collected from mice on Day 60 after mice immunized with PBS, S (10 μg per mouse; a conventional protein vaccination dose), S (15 ng/mouse; an equivalent S dose to that of DC2.4 S and DC2.4 S EBs), 2.5×10⁵ DC 2.4 cell and DC 2.4-spike cell, equivalent amount by surface area of 2.5×10⁵ DC 2.4 and DC 2.4 spike EBs.

FIG. 11 presents the results of spike IgG ELISAs from mice immunized with the IVAX-1 adjuvant. Sera was collected from mice on Day 0 (Prime). Day 14 (Booster) and Day 24 (10 days after booster) after immunization with PBS. spike protein (10 μg/mouse), equivalent spike protein of 2.5×10⁵ cells (15 ng/mouse), 2.5×10⁵ DC 2.4 cell and DC 2.4-spike cell, equivalent amount by surface area of 2.5×10⁵ DC 2.4 and DC 2.4 spike EBs.

FIG. 12 presents the results of Spike IgG ELISAs with or without IVAX-1 adjuvant injection on day 14 (booster shot) and day 24 (10 days after booster shot).

FIG. 13 provides the results of a neutralization assay obtained from mice sera immunized with IVAX-1 adjuvant. Sera and pseudotyped SARS-CoV-2 spike GFP lentivirus were incubated for 1 hour at 37° C. After incubation, 2×10⁴ ACE-2 expressing 293T cells were added and incubated for 48 hours and analyzed using flow cytometry. Mean fluorescence intensity (MFI) was used to determine the IC₅₀ of the sera sample.

FIG. 14A-C presents animal vaccination by DC2.4 S EBs, measured by antibody generation and virus neutralization. (A) Vaccination of the syngeneic C57/BL6 mice, with IVAX, and blood draw regimen (n=5). (B) Anti-S antibody in the plasma from the vaccinated mice with PBS, S (10 μg per mouse; a conventional protein vaccination dose). S (15 ng/mouse; an equivalent S dose to that of DC2.4 S and DC2 4 S EBs), DC2.4 and DC 2.4 S cells, and DC2.4 and DC2.4 S EBs (equivalent amount by surface area of 2.5×10⁵ corresponding cells) was quantified by ELISA. (C) The neutralization capability of the plasma harvested from the vaccinated mice were incubated with S-pseudotyped, GFP-expressing lentivirus before transducing 293T ACE2 cells. No antibodies were quantitated in the plasma collected at the time of prime injection (Day 0) (data now shown). *p<0.05, p<0.001, ***p<0.001 by one-way ANOVA with Tukey's post hoc test.

FIG. 15 shows IgG production with or without an adjuvant, IVAX-1. The level of IgG against S in the plasma collected from mice vaccinated with PBS, S (15 ng per mouse; an equivalent S amount to that of DC2.4 S EBs), S (10 μg per mouse; a conventional dose). 2.5×10⁵ DC2.4 cell, 2.5×10⁵ DC2.4 S cells, DC2.4 EBs, and DC2.4 S EBs, where EBs with an equivalent surface area to 2.5×10⁵ cells were used, with or without IVAX-1. The mice were immunized twice 14 days apart (prime and booster shots), and plasma were collected at the time of the booster shot (Day 14), and 10 days after the booster shot (Day 24). Data are presented as mean±SD (n=5 per group). **p<0.01, ***p<0.001 by one-way ANOVA with Tukey's post hoc test.

FIG. 16A-B demonstrates the production of IgG neutralizing S-pseudotyped lentivirus. (A) Plasma was collected at the time of the booster (Day 14) and 10 days after the booster (Day 24) from the mice vaccinated with PBS, free SPK (15 ng and 10 μg), DC2.4 and DC2.4 S cells, and DC2.4 and DC2.4 S EBs (n=5), without adjuvants, prior to (B) neutralization of S-pseudotyped lentivirus. Despite the noticeable difference in IgG amounts in the plasma collected from the mice vaccinated with S at a high dose (10 μg per mouse) and DC2.4 S EBs in Day 24 (see FIG. 15), they demonstrated comparable capabilities of virus neutralization. This indicate that EBs produce IgG with a higher neutralization capability than free proteins.

### DETAILED DESCRIPTION

As used herein and in the appended claims, the singular forms “a,” “an,” and “the” include plural referents unless the context clearly dictates otherwise. Thus, for example, reference to “an extracellular bleb” includes a plurality of such extracellular blebs and reference to “the vaccine” includes reference to one or more vaccines and equivalents thereof known to those skilled in the art, and so forth.

Also, the use of “or” means “and/or” unless stated otherwise. Similarly, “comprise,” “comprises,” “comprising” “include,” “includes,” and “including” are interchangeable and not intended to be limiting.

It is to be further understood that where descriptions of various embodiments use the term “comprising,” those skilled in the art would understand that in some specific instances, an embodiment can be alternatively described using language “consisting essentially of” or “consisting of.”

Unless defined otherwise, all technical and scientific terms used herein have the same meaning as commonly understood to one of ordinary skill in the art to which this disclosure belongs. Although many methods and reagents are similar or equivalent to those described herein, the exemplary methods and materials are disclosed herein.

All publications mentioned herein are incorporated herein by reference in full for the purpose of describing and disclosing the methodologies, which might be used in connection with the description herein. Moreover, with respect to any term that is presented in one or more publications that is similar to, or identical with, a term that has been expressly defined in this disclosure, the definition of the term as expressly provided in this disclosure will control in all respects.

It should be understood that this disclosure is not limited to the particular methodology, protocols, and reagents, etc., described herein and as such may vary. The terminology used herein is for the purpose of describing particular embodiments only and is not intended to limit the scope of the present invention, which is defined solely by the claims.

Other than in the operating examples, or where otherwise indicated, all numbers expressing quantities of ingredients or reaction conditions used herein should be understood as modified in all instances by the term “about.” The term “about” when used to described the present invention, in connection with percentages means ±1%.

The terms “blebbing”, “plasma membrane blebbing” or “cell membrane blebbing” as used herein, all refer to methods disclosed herein that induce plasma membrane blebbing in cells resulting in the production of extracellular blebs (EBs). A bleb is an irregular bulge in the plasma membrane of a cell caused by localized decoupling of the cytoskeleton from the plasma membrane. The bulge eventually separates from the parent plasma membrane taking part of the cytoplasm with it to form a vesicle, i.e., an extracellular bleb. Blebbing is also involved in some normal cell processes, including cell locomotion and cell division. While typical blebbing of the plasma membrane is a morphological feature of cells undergoing late-stage apoptosis, chemically or physically induced blebbing is an active way to convert the plasma membranes at any cellular stages with preserved cellular and molecular profiles and consistently high yields. As such, cell blebbing can be manipulated by physical or chemical treatment. It can be induced following microtubule disassembly, by inhibition of actin polymerization, increasing membrane rigidity or inactivating myosin motors, and by modulating intracellular pressure. EBs can also be produced in response to various extracellular chemical stimuli, such as exposure to agents that bind up sulfhydryl groups (i.e., sulfhydryl blocking agents).

The term “blebbing agent”, as used herein refers to chemical agents, such as sulfhydryl blocking agents, that when administered to cells induce the cells to undergo plasma membrane blebbing.

The term “extracellular bleb” or “EB” as used herein, is synonymous with an “induced cell-derived vesicle” or “ICV,” and refers to an extracellular vesicle that is formed as a direct result from contacting the cell with a blebbing agent. Accordingly, an EB is not synonymous with a naturally occurring extracellular vesicle, as the latter is formed without the presence of blebbing agent, while the former requires the use of a blebbing agent in order to be produced. The methods and compositions described herein can be applied to EBs of all sizes. In a particular embodiment, the method and compositions described herein comprise EBs that have an average diameter of 10 nm, 20 nm, 30 nm, 40 nm, 50 nm, 60 nm, 70 nm, 80 nm, 90 nm, 100 nm, 110 nm, 120 nm, 130 nm, 140 nm, 150 nm, 160 nm, 170 nm, 180 nm, 190 nm, 200 nm, 250 nm, 300 nm, 350 nm, 400 nm, 450 nm, 500 nm, 550 nm, 600 nm, 650 nm, 700 nm, 750 nm, 800 nm, 850 nm, 900 nm, 950 nm, 1000 nm, 1100 nm, 1200 nm, 1300 nm, 1400 nm, 1500 nm, 1600 nm, 1700 nm, 1800 nm, 1900 nm, 2000 nm, 2500 nm, 3000 nm, 3500 nm, 4000 nm, 5000 nm, 10 μm, 15 μm, 20 μm, 30 μm, 40 μm, 50 μm, 60 μm, 70 μm, 80 μm, 90 μm, 100 μm, or any range that includes or is between any two of the foregoing values, including fractional increments thereof. Moreover, the EBs disclosed herein are produced from genetically engineered cells or infected cells that express foreign antigen(s) or endogenous antigens that have been genetically modified for improved therapeutic outcomes. The EBs of the disclosure may further encapsulate biological molecules, such as nucleic acids, proteins, peptides, lipids, oligosaccharides, etc.; therapeutic agents, such as drug products like antivirals, antibiotics, and antifungals; prodrugs; gene silencing agents; chemotherapeutics; diagnostic agents; and components of a gene editing system, such as the CRISPR-Cas system, a CRISPRi system, or CRISPR-Cpf1 system, etc. In a particular embodiment, an EB disclosed herein is produced from a genetically engineered antigenic presenting cell or an infected cell that expresses foreign antigen(s) as disclosed herein, wherein the EB further comprises an encapsulated antiviral agent, antibiotic, and/or chemotherapeutic agent.

The term “nanometer sized extracellular bleb,” “nEB” or “nICV” as used herein, refer to EBs produced from cells using a blebbing agent as described herein having a dimeter in the nanometer size range. In a particular embodiment, the nEB has a diameter of 5 nm, 10 nm, 20 nm, 30 nm, 40 nm, 50 nm, 60 nm, 70 nm, 80 nm, 90 nm, 100 nm, 150 nm, 200 nm, 250 nm, 300 nm, 400 nm, 500 nm, 600 nm, 700 nm, 800 nm, 900 nm, up to 1000 nm, or is a range that includes or is between any two of the foregoing values, including fractional increments thereof.

The term “micrometer sized extracellular bleb”, or “mEB” or “mICV” as used herein, all refer to extracellular blebs produced from genetically engineered cells or infected cells using a blebbing agent as described herein having a dimeter in the micrometer size range. In a particular embodiment, the mEB has a diameter of 1 μm, 2 μm, 3 μm, 4 μm, 5 μm, 10 μm, 15 μm, 20 μm, 30 μm, 40 μm, 50 μm, 60 μm, 70 μm, 80 μm, 90 μm, 100 μm, or a range that includes or is between any two of the foregoing values, including fractional increments thereof.

The term “foreign antigen” as used herein, refers to an antigen that originates from outside the body. Examples of foreign antigens include parts of or substances produced by viruses or microorganisms (such as bacteria and protozoa), as well as substances in snake venom, certain proteins in foods, and components of serum and red blood cells from other individuals.

The term “endogenous antigen” refers to an antigen that originate from the subject's own cells. Examples of endogenous antigens include, but are not limited to, cancer or tumor antigens, and cellular antigens produced in result to an infection by a pathogen (e.g., bacteria, viruses, or fungi).

The term “infected cells” refers to cells that have been infected with pathogenic virus, bacterium or fungus. The extracellular blebs made from such infected cells can be used to make a vaccine preparation as disclosed herein. The pathogenic virus, bacterium or fungus may be found in its natural state or, alternatively, may be modified from its natural state.

The term “sulfhydryl blocking agent” as used herein, refers to compound or reagent that interacts with cellular sulfhydryl groups so that the sulfhydryl group is blocked or bound up by the sulfhydryl blocking agent, typically via alkylation or disulfide exchange reactions. Chemical agents that can be used in the methods or compositions disclosed herein that block or bind up sulfhydryl groups include, but are not limited to, mercury chloride, p-chloromercuribenzene sulfonic acid, auric chloride, p-chloromercuribenzoate, chlormerodrin, meralluride sodium, iodoacetamide, paraformaldehyde, dithiothreitol and N-ethylmaleimide.

The term “effective amount” as used herein, refers to an amount that is sufficient to produce at least a reproducibly detectable amount of the desired result or effect. An effective amount will vary with the specific conditions and circumstances. Such an amount can be determined by the skilled practitioner for a given situation.

The terms “patient”, “subject” and “individual” are used interchangeably herein, and refer to an animal, particularly a human, to whom treatment including prophylaxis treatment (e.g., vaccination) is provided. This includes human and non-human animals. The term “non-human animals” as used herein includes all vertebrates, e.g., mammals, such as non-human primates, (particularly higher primates), sheep, dog, rodent (e.g., mouse or rat), guinea pig, goat, pig, cat, rabbits, cows, and non-mammals such as chickens, amphibians, reptiles etc. In one embodiment, the subject is human. In another embodiment, the subject is an experimental animal or animal substitute as a disease model. “Mammal” refers to any animal classified as a mammal, including humans, non-human primates, domestic and farm animals, and zoo, sports, or pet animals, such as dogs, cats, cattle, horses, sheep, pigs, goats, rabbits, etc. Patient or subject includes any subset of the foregoing, e.g., all of the above, but excluding one or more groups or species such as humans, primates or rodents. A subject can be male or female. A subject can be a fully developed subject (e.g., an adult) or a subject undergoing the developmental process (e.g., a child, infant or fetus).

The term “isolated” when used in reference to an EB disclosed herein, refers to the fact that the EB is separated from most other cellular components from which it was generated or in which it is typically present in nature. The EBs disclosed herein are typically prepared to the state where they are substantially isolated to completely isolated from most other cellular components and cellular debris.

The term “therapeutically effective amount” as used herein, refers to an amount that is sufficient to affect a therapeutically significant reduction in one or more symptoms of the condition when administered to a typical subject who has the condition. A therapeutically significant reduction in a symptom is, e.g., about 10%, about 20%, about 30%, about 40%, about 50%, about 60%, about 70%, about 80%, about 90%, about 100%, or more as compared to a control or non-treated subject.

The term “treat” or “treatment” as used herein, refers to a therapeutic treatment wherein the object is to eliminate or lessen symptoms. Beneficial or desired clinical results include, but are not limited to, elimination of symptoms, alleviation of symptoms, diminishment of extent of condition, stabilized (i.e., not worsening) state of condition, delay or slowing of progression of the condition.

Vaccines are useful biological preparations designed to induce immune responses. In some cases, vaccines are used to increase immunity against a specific pathogen. An ideal vaccine should meet the following requirements: (1) protect not only from the disease but also prevent infection in vaccinated individuals including less immunocompromised individuals, (2) process antigenic or antigenic encoding materials and present desired antigenic materials by antigen presenting cells (APCs) to T cells, (3) elicit long-term immune responses with minimal immunizations or booster doses, and (4) have the potential for easy manufacture, storage and made accessible for worldwide vaccination at an affordable cost and limited time. Conventional vaccines involving entire organisms or large proteins leads to unnecessary antigenic load along with increased chances of allergenic responses, often requires immunogenic adjuvants for elevated immune activation and does not ensure generation of desired immune activation. Thus, novel vaccine technologies and further refinement of existing methods and strategies are required to increase the vaccine efficacy. Traditional forms of vaccines consisting of antigenic proteins have proven safety and efficacy in generating antibodies that target multiple epitopes, while inactivated and attenuated viruses are often regarded as the most potent in generating both cellular and humoral immunity. The shortcomings of the rapidly developed, currently approved mRNA and viral-vector COVID-19 vaccines include mild to critical side effects, limited efficacy, and durability against variants of concern (VOC), and challenging storage and distribution.

Vaccine antigens, especially purified or recombinant subunit vaccines, are often poorly immunogenic and require the use of adjuvants to help stimulate protective immunity. Despite the success of currently approved adjuvants, there remains a need for the development of improved adjuvants and delivery platforms that enhance protective antibody responses, especially in populations that respond poorly to vaccinations. The present disclosure provides a platform technology to induce production of extracellular blebs from engineered cells or infected cells expressing antigen(s) for vaccine applications. The cell-free, cell-mimicking platform contains specific antigens to elicit a humoral immune response without the need for adjuvants. For example, in the studies presented herein, DCs were already immune stimulated before blebbing, and the use of adjuvants only marginally improved the vaccination efficacy. Accordingly, vaccine preparations comprising EBs disclosed herein have additional safety benefits by not needing adjuvants to elicit a humoral immune response.

Genetically engineered vaccines comprising specific immunogenic fragments are more efficient in eliciting a strong and targeted immune response, and avoiding the chances of allergenic reactions. The disclosure provides for rapid and large-scale production of engineered EBs that are easily adapted to present an antigen of choice. The studies presented herein demonstrate that the EBs derived from engineered dendritic cells, maintained expression of antigenic proteins or peptides without degradation and provided a safe and effective immunogenic response in vivo. The engineered EBs are cell-free and can present antigens so as to elicit an enhanced humoral response. One of the advantages of the methods and compositions of the disclosure is that the EBs from engineered cells can maintain the antigenic dose leading to enhanced immune response and potential reduction of systemic adverse effects. Another advantage of the methods and compositions of the disclosure is that EBs from engineered cells can be used to vaccinate patients with weakened immune systems.

Provided herein are methods that can induce the production of EBs from engineered cells or infected cells by employing a unique and efficient chemically-induced production technique that initiates rapid blebbing of the cell membrane of the cells. Contrary to other extracellular vesicle production techniques, the methods of the disclosure rapidly generate high yields of induced cellular vesicles (EBs) from engineered cells that are identical in presentation to their parent cells.

An antigen presenting cell (APC) or accessory cell is a cell that displays antigen bound by major histocompatibility complex (MHC) proteins on its surface; this process is known as antigen presentation. T cells may recognize these complexes using their T cell receptors (TCRs). APCs process antigens and present them to T-cells.

Almost all cell types can present antigens in some way. They are found in a variety of tissue types. Professional antigen-presenting cells, including macrophages, B cells and dendritic cells, present foreign antigens to helper T cells, while virus-infected cells (or cancer cells) can present antigens originating inside the cell to cytotoxic T cells. In addition to the MHC family of proteins, antigen presentation relies on other specialized signaling molecules on the surfaces of both APCs and T cells.

Antigen-presenting cells are vital for effective adaptive immune response, as the functioning of both cytotoxic and helper T cells is dependent on APCs. Antigen presentation allows for specificity of adaptive immunity and can contribute to immune responses against both intracellular and extracellular pathogens. It is also involved in defense against tumors. Some cancer therapies involve the creation of artificial APCs to prime the adaptive immune system to target malignant cells.

Antigen-presenting cells fall into two categories: professional and non-professional. Those that express MHC class II molecules along with co-stimulatory molecules and pattern recognition receptors are often called professional antigen-presenting cells. The non-professional APCs express MHC class I molecules.

T cells must be activated before they can divide and perform their function. This is achieved by interacting with a professional APC which presents an antigen recognized by their T cell receptor. The APC involved in activating T cells is usually a dendritic cell. T cells cannot recognize (and therefore cannot respond to) “free” or soluble antigens. They can only recognize and respond to an antigen that has been processed and presented by cells via carrier molecules like MHC molecules. Helper T cells can recognize exogenous antigen presented on MHC class II; while, cytotoxic T cells can recognize an endogenous antigen presented on MHC class I. Most cells in the body can present an antigen to CD8+ cytotoxic T cells via MHC class I; however, the term “antigen-presenting cell” is often used specifically to describe professional APCs. Such cells express MHC class I and MHC class II molecules and can stimulate CD4+ helper T cells as well as cytotoxic T cells.

APCs can also present foreign and self-lipids to T cells and NK cells by using the CD1 family of proteins, which are structurally similar to the MHC class I family.

Professional APCs specialize in presenting antigens to T cells. They are very efficient at internalizing antigens, either by phagocytosis (e.g., macrophages), or by receptor-mediated endocytosis (B cells), processing the antigen into peptide fragments and then displaying those peptides (bound to a MHC molecule) on their membrane. The T cell recognizes and interacts with the antigen-MHC molecule complex on the membrane of the antigen-presenting cell. An additional co-stimulatory signal is then produced by the antigen-presenting cell, leading to activation of the T cell. The expression of co-stimulatory molecules and MHC class II are defining features of professional APCs. All professional APCs also express MHC class I molecules as well.

The main types of professional antigen-presenting cells are dendritic cells, macrophages, B cells, and dendritic cells.

Macrophages can be stimulated by T cell secretion of interferon. After this activation, macrophages are able to express MHC class II and co-stimulatory molecules, including the B7 complex and can present phagocytosed peptide fragments to helper T cells. Activation can assist pathogen-infected macrophages in clearing the infection. Deriving from a monocyte, type of white blood cell, they will circulate the blood and enter affected sites and differentiate from monocytes to macrophages. At the affected site, the macrophage surrounds the site of infection or tissue damage with its membrane in a mechanism called phagocytosis.

B cells can internalize antigens that bind to their B cell receptor and present it to helper T cells. Unlike T cells, B cells can recognize soluble antigens for which their B cell receptor is specific. They can then process the antigens and present peptides using MHC class II molecules. When activated by a T cell, a B cell can undergo antibody isotype switching, affinity maturation, as well as formation of memory cells.

Dendritic cells have the broadest range of antigen presentation and are necessary for activation of naïve T cells and inducing adaptive and humoral immune responses after internalizing antigens and presenting antigenic peptides on MHC molecules. DCs present antigens to both CD4+ helper and CD8+ cytotoxic T cells. They can also perform cross-presentation, a process by which they present exogenous antigen on MHC class I molecules to CD8+ cytotoxic T cells. Cross-presentation allows for the activation of these T cells. Dendritic cells also play a role in peripheral tolerance, which contributes to prevention of auto-immune disease. DCs also migrate between lymphoid and non-lymphoid tissues and modulate cytokine and chemokine gradients for inducing a durable immune response. The effective use of DCs for vaccination has not been clinically demonstrated due to inefficient antigen presentation, limited migratory capacity, and unsustainable immune stimulation in vivo, also called DC exhaustion. In overcoming the technological shortcomings of employing live DCs for vaccination, DC-derived extracellular vesicles (EVs) have demonstrated the possibility of eliciting antigen-specific neutralization of SARS-CoV-2. However, vaccine development is challenged by the contradictory immune-privileged property of EVs and by difficulties in their characterization and manufacturing arising from the high structural and functional heterogeneity, necessitating an alternative DC-mimicking vaccine.

The disclosure provides for a novel extracellular bleb (EB) vaccine platform technology that avoids the limitations of live cell and cell-derived EVs. As was shown in the studies presented herein, DCs were genetically engineered to efficiently express the spike protein(S) of SARS-CoV-2 and converted to a cell-free, DC-mimicking vaccine via chemical blebbing. Chemical blebbing creates cell-mimicking vesicles in a highly efficient, rapid, and scalable fashion, and the resulting EBs are homogenous in both structure and function. The S-expressing DC-derived EBs were used to vaccinate mice, and the collected plasma were tested for SARS-CoV-2 neutralization. The study demonstrates the utility of the EB vaccine platform technology of the disclosure. More specifically the EB vaccine platform technology of the disclosure provides for genetic engineering of cells to express desired antigens, and then presenting said antigens in a cell-free, cell-like EBs. It should be further noted that the EBs of the disclosure are stably locked to present antigens in for an extended period of time, in direct contrast to cell-based vaccines which become exhausted and protein/nucleic acid vaccines which quickly cleared.

The disclosure provides for cells (e.g., antigen presenting cells) that have been engineered to express antigens (e.g., foreign antigens or endogenous antigens that have been genetically modified for improved therapeutic outcomes). Methods to engineer cells to express antigens include, but are not limited to, non-viral vectors; viral vectors, or viruses made from thereof; introducing a transgene by using a Sleeping Beauty (SB) transposon system; and modifying the genome of the APCs by using gene editing technologies, such as CRISPR based systems, TALEN, zinc finger nucleases, etc. In a further embodiment the cells have been engineered to express antigens by introducing a viral vector into the cells. Examples of viral vectors include adenovirus vectors, lentivirus vectors, adeno-associated virus vectors, alphavirus vectors, vesicular stomatitis virus vectors, vaccinia Ankara virus vectors, Sendai virus vectors, cytomegalovirus vectors, influenza virus vectors, measles virus vectors, gammaretrovirus vectors, and spumavirus vectors. In a particular embodiment, cells are engineered to express antigens by using a lentiviral vector system.

The disclosure provides for the production and use of EBs from cells that have been infected with pathogenic virus, bacterium or fungus (e.g., EBs produced from SARS-CoV-2 infected lung epithelium cells, EBs produced from Chlamydia-infected cervical cells for Chlamydia vaccines, and more). The EBs made therefrom can be used to make a vaccine preparation as disclosed herein.

In overcoming the technological shortcomings of employing live APCs for vaccination, APC-derived extracellular vesicles (EVs) have demonstrated the possibility of eliciting antigen-specific neutralization of SARS-CoV-2. However, vaccine development is challenged by the contradictory immune-privileged property of EVs and by difficulties in their characterization and manufacturing arising from the high structural and functional heterogeneity, necessitating an alternative cell-mimicking vaccine.

The disclosure provides alternatives to live APCs and APC-derived EVs. In the studies presented herein it was shown that DCs were genetically engineered to efficiently express the spike protein(S) of SARS-CoV-2 and converted to a cell-free, DC-mimicking vaccine via chemical blebbing. Chemical blebbing created cell-mimicking vesicles in a highly efficient, rapid, and scalable fashion, and the resulting EBs that are homogenous in both structure and function. The S-expressing APC-derived EBs were used to vaccinate mice, and the collected plasma were tested for SARS-CoV-2 neutralization. This study demonstrated the feasibility of developing a novel vaccine platform that is capable of bypassing the hurdles in vaccination, including targeted uptake by APCs and antigen processing for desired antigen presentation.

EBs may be produced from cells by contacting the cells with a chemical agent that induces blebbing as further described herein. The EBs can be produced from an immortalized antigen presenting cell line, such as CB1, DC2.4, 3C10, MG38, GL7, I-11.15, 162-21.2, DEC-205, VM-2, mSXL5, 9AE10, GL1, M1/M89.18.7.HK, L11/135, M1/9.3.4.HL.2, D8/17, M1/69.16.11.H, KC-4G3, KC-4M1, M1/22.25.8.HL, XMMCO-791, PI 153/3, HO-2.2, 4D11, I-13.35, 5c8, K117, 3G5, 33D1, LCL 8664, F19, G253, LK 35.2, LS102.9, LB 27.4, M3/84.6.34, M3/38.1.2.8 HL.2, mSXL 114, UC10-4F10-11, A1G3, mSXL 18, 7E11C5, 16H3, and A20. Alternatively, the EBs can be produced from cells that have been differentiated from stem cells or progenitor cells that have been further genetically modified to express an antigen. Examples of stem cells include, human embryonic stem cells (hESCs) and induced pluripotent stem cells (iPSCs). These undifferentiated cells can be culture expanded and subsequently differentiated into APCs as described by Senju et al., Gene Therapy 18:874-883 (2011)). iPSCs- and hESCs-derived APCs and those from APC cell lines have the advantage that they can be extensively tested and characterized to maintain specific standards and offers the unique opportunity to manufacture APCs under Good

Manufacturing Practices (GMP) guidelines. Alternatively, cells can be isolated as primary cells from a multicellular organism, in particular, a human. The primary cells may be isolated and used as is, or may be grown or propagated in the laboratory for a short period of time (e.g., 10 or fewer passages, 50 or fewer passages, 100 or fewer passages). Further, the primary cell may be APCs obtained from a subject to be treated, i.e., personalized treatment. In other words, the subject is treated with EBs that are produced from the subject's own cells that have been genetically engineered to express an antigen. Additionally, the disclosure provides for the production and use of EBs from cells that have been infected with pathogenic virus, bacterium or fungus (e.g., EBs produced from SARS-CoV-2 infected lung epithelium cells. EBs produced from Chlamydia-infected cervical cells for Chlamydia vaccines, and more).

The EB production technique disclosed herein presents a scalable option for producing cell-free, cell-like vaccines that have industrial and medicinal applicability. Moreover, by using the blebbing agents described herein, genetically engineered cells can be induced to produce nano-and micro-scale EBs that can be used as vaccines. By maintaining the bioactive properties of cells, EBs can elicit an immune response when administered. In the studies presented herein, the EBs can elicit a strong immune response, even in the absence of adjuvants. Furthermore, the EBs disclosed herein can be loaded with other therapeutic agents (e.g., antibiotics, antivirals, antifungals, etc.) or adjuvants, if so desired.

As shown in the studies presented herein, EBs that were produced from genetically engineered dendritic cells to express an antigen to target SARS-CoV-2 elicited a strong immune response to SARS-CoV-2 when immunized to mice. Based upon these results, the compositions, methods and kits of the disclosure find use as (1) cell-based vaccines with defined MHC I or MHC II-restricted epitopes; and (2) cell-based vaccines consisting of multivalent epitopes. The potential advantages of the compositions, methods and kits of the disclosure include, but are not limited to, enabling modulation of the immune responses to produce more effective type of immunity for specific antigens, yielding improved antibody titers and cell-mediated immunity, broadening responses, reducing antigen dose and number of required doses.

In particular, the disclosure provides for techniques and methods that provide for high yields of EBs from genetically engineered cells or from infected cells in as little as a few hours, producing both micro and nano-scale sized EBs. For example, use of the blebbing agents described herein can induce the production of EBs in 8 h or less.

In a further embodiment, the chemical agent that induces blebbing is a sulfhydryl blocking agent. Examples of sulfhydryl blocking agents include, but are not limited to, mercury chloride, p-chloromercuribenzene sulfonic acid, auric chloride, p-chloromercuribenzoate, chlormerodrin, meralluride sodium, iodoacetmide. paraformaldehyde, dithiothreitol, and N-ethylmaleimide. In a particular embodiment, EBs are produced from blebbing induced in genetically engineered APCs by contacting the genetically engineered APCs with (1) paraformaldehyde. (2) paraformaldehyde and dithiothreitol, or (3) N-ethylmaleimide. In a further embodiment, EBs are produced from blebbing induced in genetically engineered cells by contacting the cells with a solution comprising paraformaldehyde at of 20 mM, 25 mM, 30 mM, 35 mM, 40 mM, 50 mM, 55 mM, 60 mM, 65 mM, 70 mM, 75 mM, 80 mM, 85 mM, 90 mM, 95 mM, 100 mM, 110 mM, 120 mM, 130 mM, 140 mM, 150 mM, 160 mM, 170 mM, 180 mM, 190 mM, 200 mM, 210 mM, 220 mM, 230 mM, 240 mM, 250 mM, or a range that includes any two of the foregoing concentrations, including from 20 mM and 250 mM, and from 25 mM to 50 mM.

In a yet a further embodiment, the solution comprising paraformaldehyde (PFA) further comprises dithiothreitol (DTT) at concentration of 0.2 mM, 0.4 mM, 0.5 mM, 0.6 mM, 0.8 mM, 1 mM. 1.1 mM, 1.2 mM. 1.3 mM, 1.4 mM, 1.45 mM, 1.5 mM, 1.55 mM, 1.6 mM, 1.65 mM, 1.7 mM, 1.75 mM, 1.8 mM, 1.85 mM, 1.9 mM, 1.95 mM, 2.0 mM, 2.1 mM, 2.2 mM, 2.3 mM, 2.4 mM, 2.45 mM, 2.5 mM, 2.55 mM, 2.6 mM, 2.65 mM, 2.7 mM, 2.75 mM, 2.8 mM, 2.85 mM, 2.9 mM, 2.95 mM, 3.0 mM, 3.1 mM, 3.2 mM, 3.3 mM, 3.4 mM, 3.45 mM, 3.5 mM, 3.55 mM, 3.6 mM, 3.65 mM, 3.7 mM, 3.75 mM. 3.8 mM, 3.85 mM, 3.9 mM, 3.95 mM, 4.0 mM, 4.5 mM, 5.0 mM, 5.5 mM, 6.0 mM, 6.5 mM, 7.0 mM, 7.5 mM, 8.0 mM, 8.5 mM, 9.0 mM, 9.5 mM, 10 mM, or any range that includes or is between any two of the foregoing concentrations, including from 1.0 mM to 3 mM, and 1.5 mM to 2.5 mM. In an alternate embodiment, EBs are produced from blebbing induced in genetically engineered cells by contacting the cells with a solution comprising N-ethylmaleimide (NEM) at concentration of 0.2 mM, 0.4 mM, 0.5 mM, 0.6 mM, 0.8 mM, 1.0 mM, 1.5 mM, 2.0 mM, 2.5 mM, 3.0 mM, 3.5 mM, 4.0 mM, 4.5 mM, 5.0 mM, 5.5 mM, 6.0 mM, 6.5 mM, 7.0 mM. 7.5 mM, 8.0 mM, 8.5 mM, 9.0 mM, 9.5 mM, 10.0 mM, 10.5 mM, 11.0 mM, 11.5 mM, 12 mM, 12.5 mM, 13.0 mM, 13.5 mM, 14.0 mM, 14.5 mM, 15.0 mM, 15.5 mM, 16.0 mM, 16.5 mM, 17.0 mM, 17.5 mM, 18.0 mM, 18.5 mM, 19.0 mM, 19.5 mM, 20.0 mM, or any range that includes or is between any two of the foregoing concentrations, including from 2.0 mM to 20.0 mM, and 2.0 mM to 5.0 mM. In a further embodiment, the solution comprising PFA; PFA and DTT; or NEN, comprises a buffered balanced salt solution. Examples of buffered saline solutions include but are not limited to, phosphate-buffered saline (PBS), Dulbecco's Phosphate-buffered saline (DPBS), Earles's Balanced Salt solution (EBSS), Hank's Balanced Salt Solution (HBSS), TRIS-buffered saline (TBS), and Ringer's balanced salt solution (RBSS). In a further embodiment, the solution comprising PFA; PFA and DTT; or NEM comprises a buffered balanced salt solution at a concentration/dilution of 0.5×, 0.6×, 0.7×, 0.8×, 0.9×, 1×, 2×, 3×, 4×, 5×, 6×, 7×, 8×, 9×, and 10×, or any range that includes or is between any two of the foregoing concentrations/dilutions, including fractional values thereof.

In a certain embodiment, the disclosure also provides that the EBs may be collected by any suitable means to separate EBs from APCs or cell debris of APCs. In some embodiments, to isolate EBs, cells and cell debris can be removed by centrifugation at 1000 to 1500 rpm for 1, 1.5, 2, 2.5, 3, 3.5., 4, 4.5, 5, 5.5, 6, 6.5, 7, 7.5, 8, 8.5, 9, 9.5, or 10 minutes followed by removal of APCs and cell debris of APCs. mEBs and nEBs can then be recovered by centrifugation at 10,000×g to 18,000×g for 5, 6, 7, 8, 9, 10, 11, 12, 13, 14, or 15 minutes. EBs may be further concentrated using concentrators. The size of the EBs disclosed herein could be controlled by using the isolation methods presented herein

The disclosure further provides that the EBs disclosed herein may be used (1) in combination with other agents or molecules, and/or (2) loaded with other agents or molecules, such as biological molecules, therapeutic agents, prodrugs, adjuvants, diagnostic agents, and/or components of gene editing systems. In a particular embodiment, the EBs are used in combination with or loaded with a cargo comprising one or more antivirals, antibiotics, antifungals and/or adjuvants.

EBs produced in accordance with embodiments of the disclosure may also be loaded with the cargo via direct membrane penetration. chemical labeling and conjugation, electrostatic coating, adsorption, absorption, electroporation, or any combination thereof. Further, EBs produced in accordance with certain embodiments of the disclosure may undergo multiple loading steps, such that some cargo may be introduced into the APCs prior to blebbing, while additional cargo may be loaded during or after blebbing. Additionally, EBs may be loaded with the cargo during blebbing, and further loaded with another cargo after blebbing. In a further embodiment, the EBs may be loaded with a cargo as defined above by incubating APCs or EBs with a cargo having the concentration of 25 pg/mL, 50 pg/mL, 100 pg/mL, 200 pg/mL, 300 pg/mL, 400 pg/mL, 500 pg/mL, 600 pg/mL, 700 pg/mL, 800 pg/mL, 900 pg/ml, 1 ng/mL, 10 ng/mL, 100 ng/mL, 1 μg/mL, 10 μg/mL or any range that includes or is between any two of the foregoing concentrations. Additionally, the incubation may occur for 1 hour, 2 hours, 3 hours, 4 hours, 5 hours, 6 hours, 12 hours, 24 hours, 48 hours, or any range that includes or is between any two of the foregoing time points. Alternatively, the loading conditions may occur at a ratio of EBs to a compound of 1:20 to 20:1, 1:15 to 15:1, 12:1 to 1:12, 11:1 to 1:11, 10:1 to 1:10, 9:1 to 1:9, 8:1 to 1:8, 7:1 to 1:7, 6:1 to 1:6, 5:1 to 1:5, 4:1 to 1:4. 3:1 to 1:3, 2:1 to 1:2, 1.5:1 to 1:1.5, or 1:1. Additionally, the polydispersity of cargo-loaded EBs may have a similar polydispersity index (PDI) as unloaded EBs. As such, cargo-loaded EBs may have a PDI of about 0.1, 0.2, 0.3, 0.4, 0.5, 0.6, 0.7, 0.8, 0 9, 1.0, or any range that includes or is between any two of the foregoing values.

The disclosure further provides for vaccine preparations, and pharmaceutical compositions and formulations, comprising EBs described herein for specified modes of administration. In one embodiment, a vaccine preparation or a pharmaceutical composition comprises EBs and a pharmaceutically acceptable carrier. The term “pharmaceutically acceptable carrier” as used herein means a pharmaceutically acceptable material, composition or vehicle, such as a liquid or solid filler, diluent, excipient, solvent or encapsulating material, involved in carrying or transporting the subject agents from one organ, or portion of the body, to another organ, or portion of the body. Each carrier must be “acceptable” in the sense of being compatible with the other ingredients of the composition and is compatible with administration to a subject, for example a human. Such compositions can be specifically formulated for administration via one or more of a number of routes, such as the routes of administration described herein. Supplementary active ingredients also can be incorporated into the compositions.

The disclosure further provides for the use of a pharmaceutical composition comprising EBs of the disclosure as cell-free vaccines. In a further embodiment, the disclosure also provides methods for immunizing a subject, comprising: administering an amount of EBs of the disclosure to elicit an immune response in the subject. Suitable methods of administering an EB preparation described herein to a patient include by any route of in vivo administration that is suitable for delivering EBs to a patient. The preferred routes of administration will be apparent to those of skill in the art, depending on the EB's preparation's type of vaccine used, and the target cell population. Preferred methods of in vivo administration include, but are not limited to, intravenous administration, intertumoral administration, intraperitoneal administration, intramuscular administration, intracoronary administration, intraarterial administration (e.g., into a carotid artery), subcutaneous administration, transdermal delivery, intratracheal administration, subcutaneous administration, intraarticular administration, intraventricular administration, inhalation (e.g., aerosol), intracerebral, nasal, oral, pulmonary administration, impregnation of a catheter, and direct injection into a tissue. In a particular embodiment, the EBs or a preparation comprising thereof is intramuscularly administered. In a certain embodiment, the EBs or a preparation comprising thereof is subcutaneously administered.

Intravenous, intraperitoneal, and intramuscular administrations can be performed using methods standard in the art. Aerosol (inhalation) delivery can also be performed using methods standard in the art (see, for example, Stribling et al., Proc. Natl. Acad. Sci. USA 189:11277-11281, 1992, which is incorporated herein by reference in its entirety). Oral delivery can be performed by complexing an EB preparation of the present invention to a carrier capable of withstanding degradation by digestive enzymes in the gut of an animal. Examples of such carriers include plastic capsules or tablets, such as those known in the art.

The appropriate dosage and treatment regimen for the methods of vaccination described herein will vary with respect to the EBs being delivered, and the specific condition of the subject. In one embodiment, the administration is over a period of time until the desired effect is achieved (e.g., strong immune response to an infectious agent).

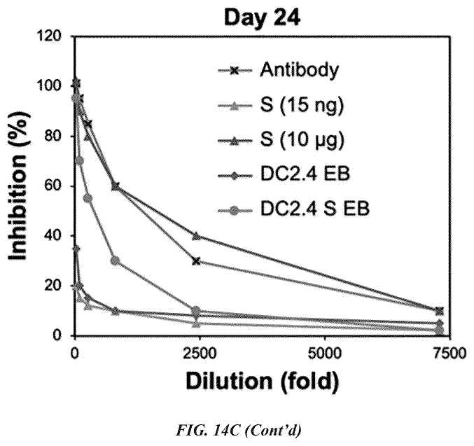

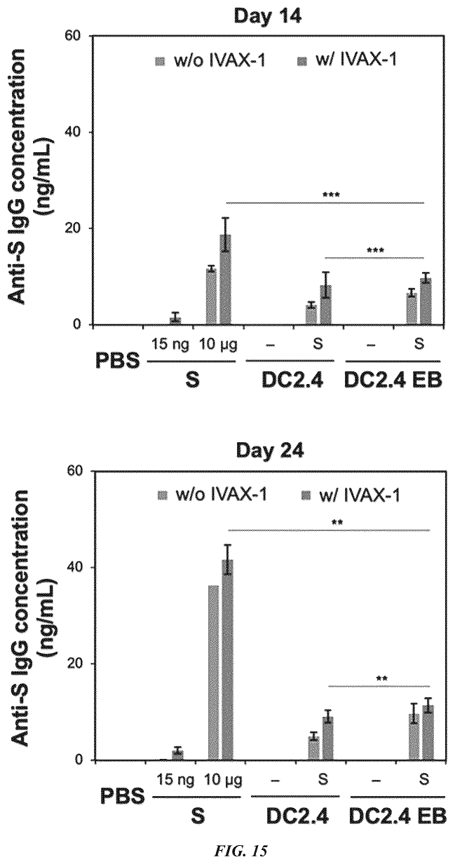

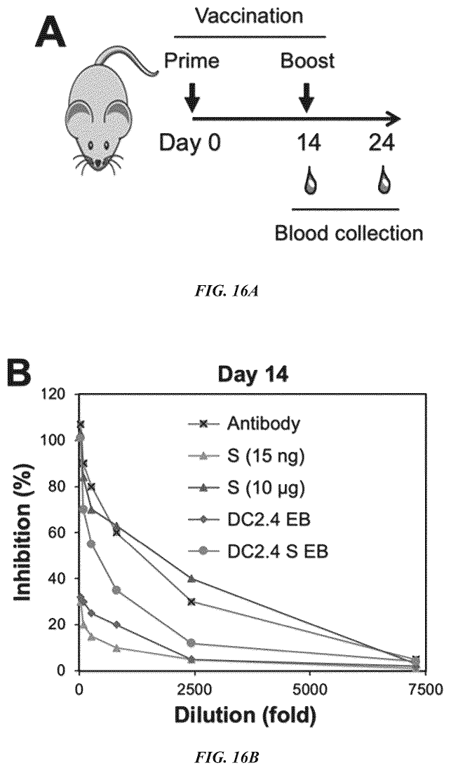

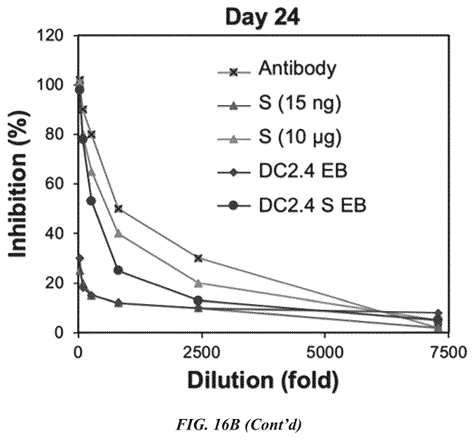

In the studies presents herein, EBs derived from S-expressing dendritic cells (DCs), substantially reduced the dose of S required to elicit a neutralizing antibody response (see FIG. 14). In addition, adjuvants were not required with the EB-based vaccine (see FIGS. 15 and 16), lowering adjuvants-caused side effects and bars for regulatory approval with simpler vaccine formulation, altogether promising for clinical translation. EB-based vaccines also offer the possibility for biologically tunable immune activation based on the maturation status of the parent DCs. In the preliminary studies presented herein, an established cell line of DC2.4 incapable of changing maturation status was used. However, the use of EBs that are derived from primary DCs or bone marrow-derived DCs with varying costimulatory signals could allow controlled levels of immune activation.

While DC-derived, S-expressing EBs generated neutralizing antibodies against S-pseudotyped virus, they might also have been able to induce S-specific cytotoxic CD8+ T cells (CTLs). According to recent evidence, cellular immunity plays a crucial role in recovering from COVID-19. Thus, DC-derived EBs were investigated as a COVID-19-treatment strategy. DC2.4 S EBs not only present antigenic peptides on MHC, but also carry the protein inside. A study reported that MHC I-loaded exosomes were poorly immunogenic. while the exosomes loaded with a full-length protein elicited strong CD8+ T cell responses in vivo. Like exosomes, the S-encapsulating EBs could have been taken up. processed. and presented by APCs, possibly resulting in directed cellular immunity. It is uncertain how much the EBs contributed to neutralizing antibody generation via direct antigen presentation to T cells vs. antigenic protein delivery. which could be further investigated by hollow EBs depleted of proteins inside.

Accordingly, the disclosure provides a new vaccine platform that mimics DCs' antigen presentation to T cells, likely along with direct antigen delivery, that addresses the limitations of current COVID-19 vaccines. The resulting EBs derived from DCs transduced to express the SARS-CoV-2 spike protein generated neutralizing antibodies comparable to levels elicited by a protein vaccine but at a much lower dose. EB-derived vaccines, which are highly stable and tolerate lyophilization, unlike currently approved COVID-19 vaccines. provides a useful strategy for preventing COVID-19 in locations where cold chain transportation and storage are unavailable.

For use in the therapeutic applications described herein, kits and articles of manufacture are also described herein. Such kits can comprise a carrier, package, or container that is compartmentalized to receive one or more containers such as vials, tubes, and the like, each of the container(s) comprising one of the separate elements to be used in a method described herein. Suitable containers include, for example, bottles, vials, syringes. and test tubes. The containers can be formed from a variety of materials such as glass or plastic.

For example, the container(s) can comprise one or more EBs described herein, optionally in a composition or in combination with another agent as disclosed herein. The container(s) optionally have a sterile access port (for example the container can be an intravenous solution bag or a vial having a stopper pierceable by a hypodermic injection needle). Such kits optionally comprise a compound disclosed herein with an identifying description or label or instructions relating to its use in the methods described herein.

A kit will typically comprise one or more additional containers, each with one or more of various materials (such as reagents, optionally in concentrated form, and/or devices) desirable from a commercial and user standpoint for use of a compound described herein. Non-limiting examples of such materials include, but are not limited to, buffers, diluents, filters, needles, syringes; carrier, package, container, vial and/or tube labels listing contents and/or instructions for use, and package inserts with instructions for use. A set of instructions will also typically be included.

A label can be on or associated with the container. A label can be on a container when letters, numbers or other characters forming the label are attached, molded or etched into the container itself; a label can be associated with a container when it is present within a receptacle or carrier that also holds the container, e.g., as a package insert. A label can be used to indicate that the contents are to be used for a specific application. The label can also indicate directions for use of the contents, such as in the methods described herein.

The disclosure further provides that the methods and compositions described herein can be further defined by the following aspects (aspects 1 to 41):
1. A vaccine preparation comprising extracellular blebs from a cell that has been genetically engineered to express an antigen(s),
   wherein the extracellular blebs are produced from the cell by treating the antigen presenting cell with a blebbing agent, and
   wherein the antigen is displayed on the surface of the extracellular blebs.
2. The vaccine preparation of aspect 1, wherein the cell is selected from a macrophage, a B cell and a dendritic cell.
3. The vaccine preparation of aspect 1 or aspect 2, wherein the cell is a dendritic cell.
4. The vaccine preparation of any one of the preceding aspects, wherein the cell is an immortalized cell.
5. The vaccine preparation of any one of the preceding aspects, wherein the cell is differentiated from human embryonic stem cells (hESCs) or induced pluripotent stem cells (iPSCs) from a human subject.
6. The vaccine preparation of any one of aspect 1 to 4, wherein the cell is a human primary cell.
7. The vaccine preparation of any one of the preceding aspects, wherein the antigen(s) is a foreign antigen(s) or an endogenous antigen that has been genetically modified for an improved therapeutic outcome.
8. The vaccine preparation of aspect 7, wherein the foreign antigen(s) is from a pathogenic or disease-causing microorganism.
9. The vaccine preparation of aspect 8, wherein the pathogenic or disease-causing microorganism is a bacterium, a fungus, or a virus.
10. The vaccine preparation of aspect 9, wherein the bacterium is selected from Actinomyces israelii, Bacillus anthracis, Bacillus cereus, Bartonella henselae, Bartonella quintana, Bordetella pertussis, Borrelia burgdorferi, Borrelia garinii, Borrelia afzelii, Borrelia recurrentis, Brucella abortus, Brucella canis, Brucella melitensis, Brucella suis, Campylobacter jejuni, Chlamydia pneumoniae, Chlamydia trachomatis, Chlamydophila psittaci, Clostridium botulinum, Clostridium difficile, Clostridium perfringens, Clostridium tetani, Corynebacterium diphtheriae, Enterococcus faecalis, Enterococcus faecium, Escherichia coli, Francisella tularensis, Haemophilus influenzae, Helicobacter pylori, Legionella pneumophila, Leptospira interrogans, Leptospira santarosai, Leptospira weilii, Leptospira noguchii, Listeria monocytogenes, Mycobacterium leprae, Mycobacterium tuberculosis, Mycobacterium ulcerans, Mycoplasma pneumoniae, Neisseria gonorrhoeae, Neisseria meningitidis, Pseudomonas aeruginosa, Rickettsia rickettsia, Salmonella typhi, Salmonella typhimurium, Shigella sonnet, Staphylococcus aureus, Staphylococcus epidermidis, Staphylococcus saprophyticus, Streptococcus agalactiae, Streptococcus pneumoniae, Streptococcus pyogenes, Treponema pallidum, Ureaplasma urealyticum, Vibrio cholerae, Yersinia pestis, Yersinia enterocolitica, and Yersinia pseudotuberculosis.
11. The vaccine preparation of aspect 9, wherein the fungus is selected from Absidia corymbifera, Absidia ramose, Achorion gallinae, Actinomadura spp., Ajellomyces dermatididis, Aleurisma brasiliensis, Allersheria boydii, Arthroderma spp., Aspergillus flavus, Aspergillus fumigatu, Basidiobolus spp, Blastomyces spp, Cadophora spp, Candida albicans, Cercospora apii, Chrysosporium spp, Cladosporium spp, Cladothrix asteroids, Coccidioides immitis, Cryptococcus albidus, Cryptococcus gattii, Cryptococcus laurentii, Cryptococcus neoformans, Cunninghamella elegans, Dematium wernecke, Discomyces israelii, Emmonsia spp, Emmonsiella capsulate, Endomyces geotrichum, Entomophthora coronate, Epidermophyton floccosum, Filobasidiella neoformans, Fonsecaea spp., Geotrichum candidum, Glenospora khartoumensis, Gymnoascus gypseus, Haplosporangium parvum, Histoplasma, Histoplasma capsulatum, Hormiscium dermatididis, Hormodendrumspp., Keratinomyces spp, Langeronia soudanense, Leptosphaeria senegalensis, Lichtheimia corymbifera, Lobmyces loboi., Loboa loboi, Lobomycosis, Madurella spp., Malassezia furfur, Micrococcus pelletieri, Microsporum spp, Monilia spp., Mucor spp., Mycobacterium tuberculosis, Nannizzia spp., Neotestudina rosatii, Nocardia spp., Oidium albicans, Oospora lactis, Paracoccidioides brasiliensis, Petriellidium boydii, Phialophora spp., Piedraia hortac, Pityrosporum furfur, Pneumocystis jirovecii (or Pneumocystis carinii), Pulldaria gougerotii, Pyrenochaeta romeroi, Rhinosporidium seeberi, Sabouraudites (Microsporum), Sartorya fumigate, Sepedonium, Sporotrichum spp., Stachybotrys, Stachybotrys chartarum, Streptomyce spp., Tinea spp., Torula spp, Trichophyton spp, Trichosporon spp, and Zopfia rosatii.
12. The vaccine preparation of aspect 9, wherein the virus is selected from the group consisting of Adeno-associated virus, Aichi virus, Australian bat lyssavirus, BK polyomavirus, Banna virus, Barmah forest virus, Bunyamwera virus, Bunyavirus La Crosse, Bunyavirus snowshoe hare, Cercopithecine herpesvirus, Chandipura virus, Chikungunya virus, Cosavirus A, Coronavirus, Cowpox virus, Coxsackievirus, Crimean-Congo hemorrhagic fever virus, Dengue virus, Dhori virus, Dugbe virus, Duvenhage virus, Eastern equine encephalitis virus, Ebolavirus, Echovirus, Encephalomyocarditis virus, Epstein-Barr virus, European bat lyssavirusalitis, GB virus C/Hepatitis G virus Pegivirus, Hantan virus, Hendra virus, Hepatitis A virus, Hepatitis B virus, Hepatitis C virus, Hepatitis E virus, Hepatitis delta virus, Horsepox virus, Human adenovirus, Human astrovirus, Human coronavirus, Human cytomegalovirus, Human enterovirus, Human herpesvirus, Human immunodeficiency virus, Human papillomavirus, Human parainfluenza, Human parvovirus B19, Human respiratory syncytial virus, Human rhinovirus, Human SARS coronavirus, Human spumaretrovirus, Human T-lymphotropic virus, Human torovirus, Influenza A virus, Influenza B virus, Isfahan virus, JC polyomavirus, Japanese encephalitis virus, Junin arenavirus, KI Polyomavirus, Kunjin virus, Lagos bat virus, Lake Victoria Marburgvirus, Langat virus, Lassa virus, Lordsdale virus, Louping ill virus, Lymphocytic choriomeningitis virus, Machupo virus, Mayaro virus, MERS coronavirus, Measles virus, Mengo encephalomyocarditis virus, Merkel cell polyomavirus, Mokola virus, Molluscum contagiosum virus, Monkeypox virus, Mumps virus, Murray valley encephalitis virus, New York virus, Nipah virus, Norwalk virus, O'nyong-nyong virus, Orf virus, Oropouche virus, Pichinde virus, Poliovirus, Punta toro phlebovirus, Puumala virus, Rabies virus, Rift valley fever virus, Rosavirus A, Ross river virus, Rotavirus A, Rotavirus B, Rotavirus C, Rubella virus, Sagiyama virus, Salivirus A, Sandfly fever sicilian virus, Sapporo virus, Semliki forest virus, Seoul virus, Simian foamy virus, Simian virus, Sindbis virus, Southampton virus, St. louis encephalitis virus, Tick-borne powassan virus, Torque teno virus, Toscana virus, Uukuniemi virus, Vaccinia virus, Varicella-zoster virus, Variola virus O, Venezuelan equine encephalitis virus, Vesicular stomatitis virus, Western equine encephalitis virus, WU polyomavirus, West Nile virus, Yaba monkey tumor virus, Yaba-like disease virus, Yellow fever virus, and Zika virus.
13. The vaccine preparation of aspect 12, wherein the human coronavirus is SARS-CoV-2.
14. The vaccine preparation of any one of aspects 1 to 6, wherein the antigen is a cancer or tumor antigen, preferably where the cancer or tumor antigen is selected from CD19, BCMA, alpha-fetoprotein, cancer antigen 125, cancer antigen 15-3, carbohydrate antigen 19-9, carcinoembryonic antigen, human chorionic gonadotropin, and prostate-specific antigen.
15. The vaccine preparation of any one of the preceding aspects, wherein a viral vector is used to genetically engineer the antigen presenting cell to expresses the antigen(s).
16. The vaccine preparation of aspect 15, wherein the viral vector is a lentivirus vector, an adenovirus vector, an adeno-associated virus vector, or a gammaretrovirus vector.
17. The vaccine preparation of aspect 16, wherein the viral vector is a lentivirus vector.
18. The vaccine preparation of any one of the preceding aspects, wherein the vaccine preparation further comprises an adjuvant, preferably wherein the adjuvant is selected from aluminum hydroxide, aluminum phosphate, aluminum potassium sulfate, AS04, MF59, ASO1_{B}, CpG 1018, and IVAX-1.
19. The vaccine preparation of any one of aspects 1 to 17, wherein the vaccine preparation does not comprise an adjuvant.
20. The vaccine preparation of any one of the preceding aspects, wherein the extracellular blebs comprise one or more of the following surface and maturation markers CD11c, MHC I, CD40, CD80, and/or CD86, preferably wherein the extracellular blebs comprise CD11c, MHC I, CD40, CD80, and CD86 surface and maturation markers.
21. A method of making the vaccine preparation of any one of the preceding aspects, comprising:
   generating extracellular blebs from a genetically engineered cell by contacting the cell with the one or more sulfhydryl blocking agents for 3 min to 24 h; and
   isolating the extracellular blebs.
22. The method of aspect 21, wherein the one or more sulfhydryl blocking agents are selected from the group consisting of mercury chloride, p-chloromercuribenzene sulfonic acid, auric chloride, p-chloromercuribenzoate, chlormerodrin, meralluride sodium, iodoacetmide, paraformaldehyde, dithiothreitol, and N-ethylmaleimide.
23. The method of aspect 21 or aspect 22, wherein the one or more sulfhydryl blocking agents is N-ethylmaleimide.
24. The method of aspect 23, wherein N-ethylmaleimide is used at a concentration of 0.2 mM to 30 mM.
25. The method of any one of aspects 21 to 24, wherein the cell is selected from a macrophage, a B cell and a dendritic cell.
26. The method of any one of aspects 21 to 25, wherein the cell is a dendritic cell.
27. The method of any one of aspects 21 to 26, wherein the cell is an immortalized cell.
28. The method of any one of aspects 21 to 27, wherein the cell is differentiated from human embryonic stem cells (hESCs) or induced pluripotent stem cells (iPSCs) from a human subject.
29. The method of any one of aspects 21 to 27, wherein the cell is a human primary cell.
30. The method of any one of aspects 21 to 29, wherein the antigen(s) is a foreign antigen(s).
31. The method of aspect 30, wherein the foreign antigen(s) is from a pathogenic or disease-causing microorganism.
32. The method of aspect 31, wherein the pathogenic or disease-causing microorganism is a bacterium, a fungus, or a virus.
33. The method of aspect 32, wherein the bacterium is selected from Actinomyces israelii, Bacillus anthracis, Bacillus cereus, Bartonella henselae, Bartonella quintana, Bordetella pertussis, Borrelia burgdorferi, Borrelia garinii, Borrelia afzelii, Borrelia recurrentis, Brucella abortus, Brucella canis, Brucella melitensis, Brucella suis, Campylobacter jejuni, Chlamydia pneumoniae, Chlamydia trachomatis, Chlamydophila psittaci, Clostridium botulinum, Clostridium difficile, Clostridium perfringens, Clostridium tetani, Corynebacterium diphtheriae, Enterococcus faecalis, Enterococcus faecium, Escherichia coli, Francisella tularensis, Haemophilus influenzae, Helicobacter pylori, Legionella pneumophila, Leptospira interrogans, Leptospira santarosai, Leptospira weilii, Leptospira noguchii, Listeria monocytogenes, Mycobacterium leprae, Mycobacterium tuberculosis, Mycobacterium ulcerans, Mycoplasma pneumoniae, Neisseria gonorrhoeae, Neisseria meningitidis, Pseudomonas aeruginosa, Rickettsia rickettsia, Salmonella typhi, Salmonella typhimurium, Shigella sonnei, Staphylococcus aureus, Staphylococcus epidermidis, Staphylococcus saprophyticus, Streptococcus agalactiae, Streptococcus pneumoniae, Streptococcus pyogenes, Treponema pallidum, Ureaplasma urealyticum, Vibrio cholerae, Yersinia pestis, Yersinia enterocolitica, and Yersinia pseudotuberculosis.
34. The method of aspect 32, wherein the fungus is selected from Absidia corymbifera, Absidia ramose, Achorion gallinae, Actinomadura spp., Ajellomyces dermatididis, Aleurisma brasiliensis, Allersheria boydit, Arthroderma spp., Aspergillus flavus, Aspergillus fumigatu, Basidiobolus spp, Blastomyces spp, Cadophora spp, Candida albicans, Cercospora apii, Chrysosporium spp, Cladosporium spp, Cladothrix asteroids, Coccidioides immitis, Cryptococcus albidus, Cryptococcus gattii, Cryptococcus laurentii, Cryptococcus neoformans, Cunninghamella elegans, Dematium wernecke, Discomyces israelii, Emmonsia spp, Emmonsiella capsulate, Endomyces geotrichum, Entomophthora coronate, Epidermophyton floccosum, Filobasidiella neoformans, Fonsecaea spp., Geotrichum candidum, Glenospora khartoumensis, Gymnoascus gypseus, Haplosporangium parvum, Histoplasma, Histoplasma capsulatum, Hormiscium dermatididis, Hormodendrumspp., Keratinomyces spp, Langeronia soudanense, Leptosphaeria senegalensis, Lichtheimia corymbifera, Lobmyces loboi., Loboa loboi, Lobomycosis, Madurella spp., Malassezia furfur, Micrococcus pelletieri, Microsporum spp, Monilia spp., Mucor spp., Mycobacterium tuberculosis, Nannizzia spp., Neotestudina rosatii, Nocardia spp., Oidium albicans, Oospora lactis, Paracoccidioides brasiliensis, Petriellidium boydii, Phialophora spp., Piedraia hortae, Pityrosporum furfur, Pneumocystis jirovecii (or Pneumocystis carinii), Pullularia gougerotil, Pyrenochaeta romeroi, Rhinosporidium seeberi, Sabouraudites (Microsporum), Sartorya fumigate, Sepedonium, Sporotrichum spp., Stachybotrys, Stachybotrys chartarum, Streptomyce spp., Tinea spp., Torula spp. Trichophyton spp, Trichosporon spp, and Zopfia rosatii.
35. The method of aspect 32, wherein the virus is selected from the group consisting of Adeno-associated virus, Aichi virus, Australian bat lyssavirus, BK polyomavirus, Banna virus, Barmah forest virus, Bunyamwera virus, Bunyavirus La Crosse, Bunyavirus snowshoe hare, Cercopithecine herpesvirus, Chandipura virus, Chikungunya virus, Cosavirus A, Coronavirus, Cowpox virus, Coxsackievirus, Crimean-Congo hemorrhagic fever virus, Dengue virus, Dhori virus, Dugbe virus, Duvenhage virus, Eastern equine encephalitis virus, Ebolavirus, Echovirus, Encephalomyocarditis virus, Epstein-Barr virus, European bat lyssavirusalitis. GB virus C/Hepatitis G virus Pegivirus, Hantan virus, Hendra virus, Hepatitis A virus, Hepatitis B virus, Hepatitis C virus, Hepatitis E virus, Hepatitis delta virus, Horsepox virus, Human adenovirus, Human astrovirus, Human coronavirus, Human cytomegalovirus, Human enterovirus, Human herpesvirus, Human immunodeficiency virus, Human papillomavirus, Human parainfluenza, Human parvovirus B19, Human respiratory syncytial virus, Human rhinovirus, Human SARS coronavirus, Human spumaretrovirus, Human T-lymphotropic virus, Human torovirus, Influenza A virus, Influenza B virus, Isfahan virus, JC polyomavirus, Japanese encephalitis virus, Junin arenavirus, KI Polyomavirus, Kunjin virus, Lagos bat virus, Lake Victoria Marburgvirus, Langat virus, Lassa virus, Lordsdale virus, Louping ill virus, Lymphocytic choriomeningitis virus, Machupo virus, Mayaro virus, MERS coronavirus, Measles virus, Mengo encephalomyocarditis virus, Merkel cell polyomavirus, Mokola virus, Molluscum contagiosum virus, Monkeypox virus, Mumps virus, Murray valley encephalitis virus, New York virus, Nipah virus, Norwalk virus, O'nyong-nyong virus, Orf virus, Oropouche virus, Pichinde virus, Poliovirus, Punta toro phlebovirus, Puumala virus, Rabies virus, Rift valley fever virus, Rosavirus A, Ross river virus, Rotavirus A, Rotavirus B, Rotavirus C, Rubella virus, Sagiyama virus, Salivirus A, Sandfly fever sicilian virus, Sapporo virus, Semliki forest virus, Seoul virus, Simian foamy virus, Simian virus, Sindbis virus, Southampton virus, St. louis encephalitis virus, Tick-borne powassan virus, Torque teno virus, Toscana virus, Uukuniemi virus, Vaccinia virus, Varicella-zoster virus, Variola virus O, Venezuelan equine encephalitis virus, Vesicular stomatitis virus, Western equine encephalitis virus, WU polyomavirus, West Nile virus, Yaba monkey tumor virus, Yaba-like disease virus, Yellow fever virus, and Zika virus.
36. The method of aspect 35, wherein the human coronavirus is SARS-CoV-2.
37. The method of any one of aspects 21 to 29, wherein the antigen is a cancer or tumor antigen, preferably where the cancer or tumor antigen is selected from CD19, BCMA, alpha-fetoprotein, cancer antigen 125, cancer antigen 15-3, carbohydrate antigen 19-9, carcinoembryonic antigen, human chorionic gonadotropin, and prostate-specific antigen.
38. The method of any one of aspects 21 to 36, wherein the genetically engineered antigen presenting cells are made by transforming the antigen presenting cells with a viral rector that encodes the antigen(s).
39. The method of aspect 38. wherein the viral vector is a lentivirus vector, an adenovirus vector, an adeno-associated virus vector, or a gammaretrovirus vector.
40. The method of aspect 39, wherein the viral vector is a lentivirus vector.
41. A method of immunizing a subject, comprising administering a therapeutically effective amount of the vaccine preparation of any one of aspects 1 to 20 to the subject.

The following examples are intended to illustrate but not limit the disclosure. While they are typical of those that might be used, other procedures known to those skilled in the art may alternatively be used.

### EXAMPLES

Preparation of SARS-CoV-2 spike protein-expressing cells. The mouse dendritic cell (DC) line, DC2.4, (ATCC, Manassas, VA) was cultured in high glucose DMEM supplemented with 10% (v/v) FBS and penicillin-streptomycin (100 U/mL), all from Thermo Fisher Scientific (Waltham, MA), at 37° C. with 5% CO₂ and passaged every other day. The cells were plated at a density of 5×10⁵/well in a 6-well for 24 h and then transduced with 2×10⁶ TU/well of SARS-CoV-2 spike protein (S alpha)-encoding lentivirus (BPS bioscience, San Diego, CA) in the presence of 5 μg/ml of polybrene (Thermo Fisher Scientific). After 72 h of transduction. the cells were selected by media containing 0.5 μg/mL of puromycin (Thermo Fisher Scientific) for an additional 2 weeks. The S expression in the resulting DC2.4 S cells was analyzed by flow cytometry after staining with anti-SI primary antibody (BPS Bioscience) and FITC-conjugated goat anti-human IgG secondary antibody (Thermo Fisher Scientific).

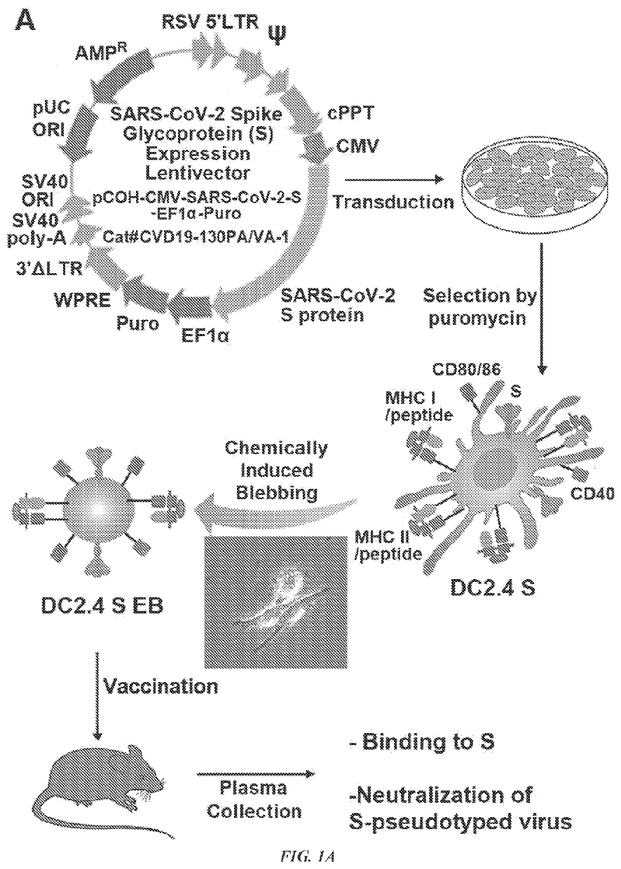

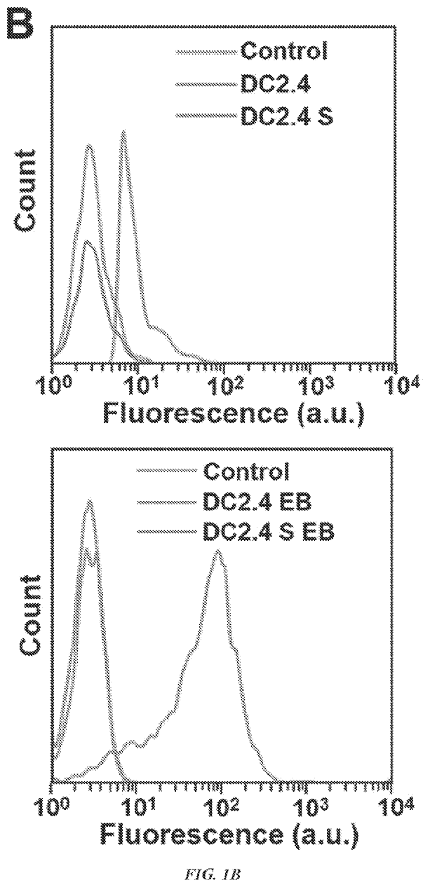

Lentiviral transduction of dendritic cell. FIG. 1 shows a schematic illustration for SARS-CoV-2 spike-encoding lentiviral transduction in a dendritic cell line (DC2.4 cell line). The lentivirus was transduced at MOI of 50 in presence of 7 μg/mL of polybrene in serum free media for 4 hours, every 30 minutes the plates were swirled to enhance transduction efficiency. SARS-CoV-2 spike glycoprotein lentivirus consists of a puromycin resistance marker and 48 hours after transduction. spike expressing transduced cells were selected using puromycin at 5 g/mL for 2 weeks. Media containing puromycin was changed every 2 days.

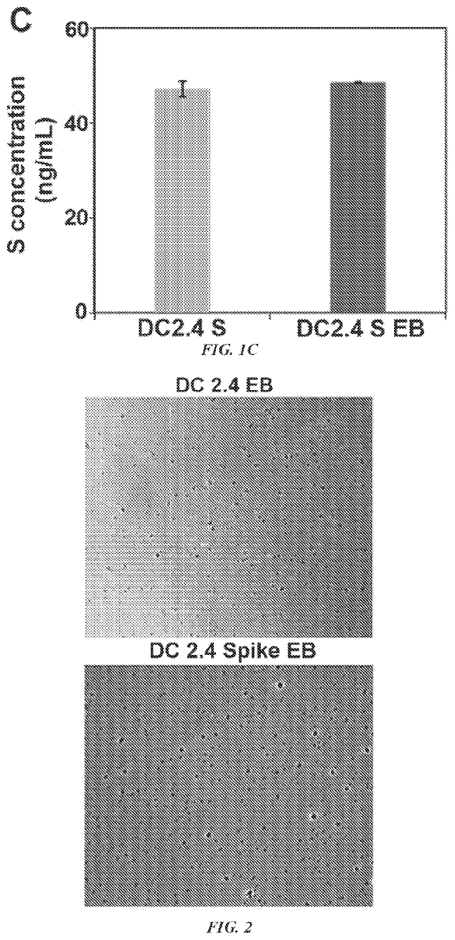

Production, collection, and characterization of DC-derived extracellular blebs (EBs).). N-Ethylmaleimide (NEM, Thermo Fisher Scientific) stock solution was prepared by dissolving NEM at 37° C. in 10 mL DI water to a concentration of 2 mM, and sterile filtered using a 0.22 μM syringe filter. Blebbing buffer containing 0.22 mM NEM by adding 90 μL of NEM stock solution to 10 mL DPBS was prepared immediately before use. DC2.4 or DC2.4 S cells (2.5×10⁵ cells) were washed 3 times in warm DPBS and incubated in the blebbing buffer for 6 h at 37° C. and 5% CO₂ to produced micro-sized EBs. The supernatant was collected and centrifuged at 1,000×g for 5 min to pellet cells and cell debris, followed by another centrifugation of supernatant at 16,100×g for 10 min. The collected DC2.4 and DC2.4 S EBs were further washed 3 times with 1×DPBS via repeated centrifugation 16,100×g for 10 min to remove any residual blebbing reagents. The DC2.4 and DC2.4 S EB pellets were finally resuspended in 1×DPBS and confirmed to be free of cells or cell debris and debris under a microscope. The resulting blebs were imaged using a light microscopy and representative images were taken and showed homogenous blebs in both dendritic cells and dendritic cells expressing spike protein (see FIG. 2). The S expression on the surface of the EBs was analyzed by flow cytometry as described for DC2.4 S cells earlier. The surface areas of the EBs were compared with those of the corresponding DC2.4 cells. Briefly, the membrane of an equal number of DC2.4 and DC2.4 S cells was stained with PKH26 according to the manufacturer's instructions (Thermo Fisher Scientific), followed by blebbing to EBs as described earlier. The PKH26-stained DC2.4 and DC2.4 S cells and DC2.4 or DC2.4 S EBs were lysed in RIPA buffer with gentle vortexing to obtain homogenized membranes. The resulting lysates were analyzed for fluorescence at 550 nm Ex/570 nm Em using a BioTek Synergy plate reader (Agilent, Santa Clara, CA), and their fluorescence was compared using an equal amount by surface area.

Quantitating Spike expression on dendritic cell line. 2 weeks after puromycin selection, spike expressing DC2.4 cells (DC2.4 S cells) were analyzed using flow cytometry. Cells were grown to 80% confluency in RPMI media supplemented with 10% FBS and 1% Pen-strep. Confluent cells were collected and 1×106 cells were labeled with anti-spike antibody for 1 hour in antibody binding buffer and unbound antibody were washed 3× at 300×g for 5 minutes. Spike labeled cells were conjugated with Alexa fluor 488-anti-rabbit IgG (H+L), F(ab′)₂ fragment for 30 minutes and analyzed using flow cytometry.

In vivo vaccination, antibody quantification, and virus neutralization. All animal work was reviewed and approved by the UCI Institutional Animal Care and Use Committee (IACUC protocol #AUP-20-116). Female 7-10-week-old C57BL/6 mice (Charles River Laboratories. Wilmington, MA) were subcutaneously injected with 100 μL of 1×PBS. S (10 μg per mouse; a conventional protein vaccination dose), S (15 ng/mouse; an equivalent S dose to that of DC2.4 S and DC2.4 S EBs), 2.5×105 DC2.4 or DC2.4 S cells, or DC2.4 or DC2.4 S EBs at an equivalent surface area to DC2.4 and DC2.4 S cells along with IVAX containing 1 nmole MPLA and 3 nmole CpG-1018 in sterile PBS, mixed with an equal volume of AddaVax™ (Front Immunol. 2021:12: 692151). The mice received a priming injection on Day 0 and a booster injection on Day 14. Immediately before and 10 days after the booster injection (Day 14 and 24), blood was collected into heparinized microcapillary tubes from the saphenous vein. After centrifugation at 6,000 kg for 15 min. the resulting plasma was tested for specific binding to SARS-CoV-2 spike proteins by ELISA. Briefly, recombinant SARS-CoV-2 spike protein (Raybiotech, Peachtree Corners, GA) was coated at 2 μg in 100 μL coating buffer (Thermo Fisher Scientific) per well of a 96-well plate overnight. The plates were blocked by adding 100 μL per well of blocking buffer consisting of 5% (w/v) non-fat dry milk in DPBS containing 0.05% (w/v) Tween-20 and incubated at room temperature for 2 h. The plate was then rinsed three times using an ELISA wash buffer (Thermo Fisher Scientific). The plasma samples were diluted 20-fold in 150 μL 1×ELISA diluent buffer was added to the wells and incubated for 2 h at room temperature (RT). The plates were washed three times using an ELISA wash buffer, and the detection antibody (mouse IgG-HRP [H+L], Waltham, MA) was added at the manufacture's recommended concentration followed by an additional 1 h incubation at RT. After the plate was washed five times, 100 μL of TMB substrate solution (Thermo Fisher Scientific) was added to each well before incubation for 15 min at RT. The reaction was stopped by 100 μL ELISA stop solution (Thermo Fisher Scientific) per well and the absorbance was measured at 450 nm and 570 nm using a SpectraMax Plus plate reader (Molecular Devices, USA). The absorbance reading at 570 nm was subtracted by that at 450 nm for optical correction. The antibody concentration in the plasma samples was quantified by comparing the calibration curve of the standard. For the virus neutralization assay. 100 μL of serially diluted plasma was incubated with 100 μL of 1×10⁴ GFP-expressing lentivirus pseudo-typed with SARS-CoV-2 spike alpha protein (BPS bioscience. San Diego, CA) in DMEM media supplemented with 10% FBS and 1% penicillin-streptomycin for 1 h at 37° C. followed by the addition of 1×10⁴ HEK 293T cells expressing human angiotensin-converting enzyme 2 (hACE2) (BEI Resources [NR-52511], NIAID/NIH) and incubated for 48 h. For reference, anti-S antibodies, named COVA1-18, gifted from Marit J. van Gils (University of Amsterdam) were used after a series of dilutions from the highest concentration of 1 μg/mL. The cells were analyzed for GFP expression using flow cytometry, and the relative transduction inhibition was determined by mean GFP fluorescence intensity.

Statistical Analysis. For all in vitro studies, triplicate data were analyzed. To achieve statistical significance in in vivo studies, 5 animals per treatment group (n=5) were used. Two-tailed Student's t-test (GraphPad Prism Ver. 8) was used to calculate the statistical significance of comparisons between two groups. and p-values less than 0.05 were considered significant. S antibody quantification by ELISA and S-pseudotyped lentivirus neutralization assays were analyzed using one-way analysis of variance (ANOVA) with Tukey's post-hoc test for multiple comparisons between subgroups.

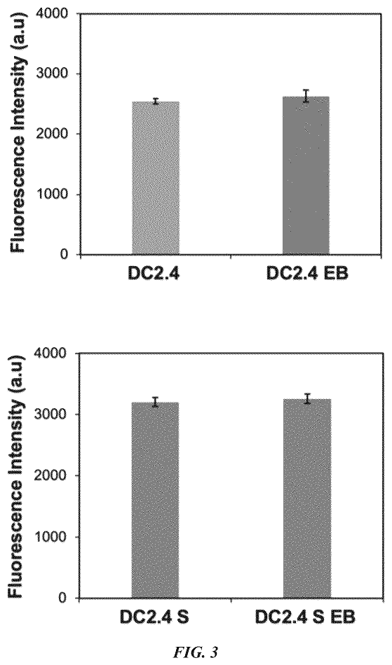

Spike expression in extracellular blebs derived from spike expressing dendritic cells. The moderate S expression by DC2.4 S cells, which was preserved on DC2.4 S EBs, was confirmed by flow cytometry (see FIG. 1B). Notably, S expression on DC2.4 S EBs was higher than that on DC2.4 S cells, possibly due to non-specific optical background signal of the smaller DC2.4 S EBs than DC2.4 S cells for the same S density on the surface, as evidenced by the equivalent S contents in the lysates (see FIG. 1C). The highly efficient conversion of DC2.4 and DC2.4 S cells to the corresponding EBs was confirmed by comparing the fluorescent lysates of PKH26-labeled cells and the resulting EBs (see FIG. 3).

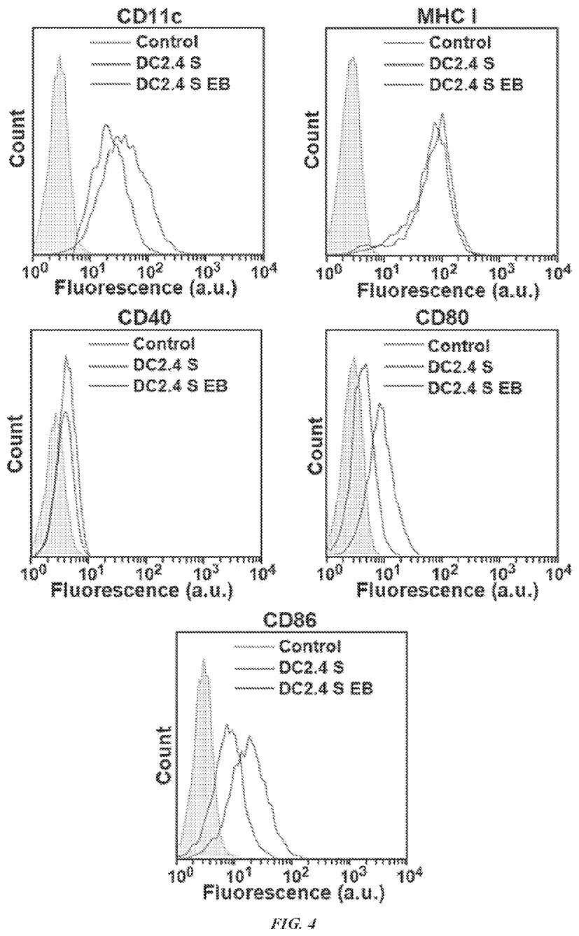

Dendritic cell surface and maturation marker expression in extracellular blebs derived from spike expressing dendritic cells. In addition to the S expression, DC surface and maturation markers, CD11c, MHC I, CD40, CD80, and CD86, were found to be comparable between DC2.4 and DC2.4 S cells and their corresponding EBs when characterized by flow cytometry (see FIG. 4), although EBs showed slightly higher signals than the cells for CD11c, CD80, and CD86, likely for the reason described for S expression quantification. The results therefore confirmed highly efficient preparation of S-expressing DCs from which EBs closely mimicking their molecular profiles were produced.

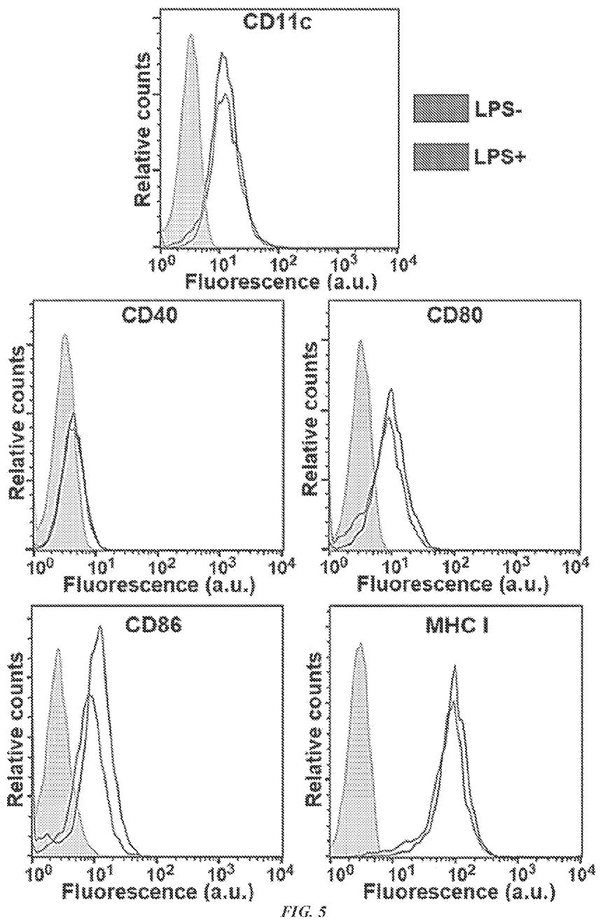

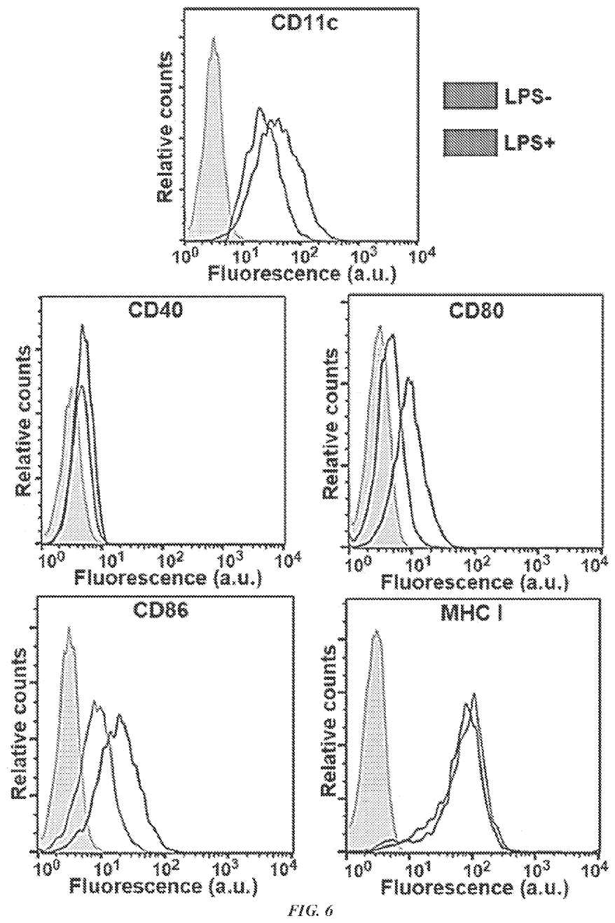

Effect of lipopolysaccharide on dendritic cell surface and maturation marker expression in extracellular blebs derived from spike expressing dendritic cells. DC maturations express high levels of co-stimulatory molecules and immunostimulatory cytokines, which indicates that DCs are phenotypically and functionally in a mature state Lipopolysaccharide (LPS) is known to modulate the phenotype of dendritic cells. To determine if LPS modulates DC phenotype in vitro, DC 2.4 and DC 2.4 Spike cells were incubated with LPS at 20 ng/mL for 24 hours. Co-stimulatory molecules using fluorescence labeled antibodies against CD11c, CD40, CD80, CD86 and MHC I were analyzed using flow cytometry. 1×10⁶ cells were labeled with the mentioned fluorescence antibodies. LPS-induced maturation did not change the molecular presentation in both the DC 2.4 (see FIG. 5) and DC 2.4 Spike cell line (see FIG. 6).

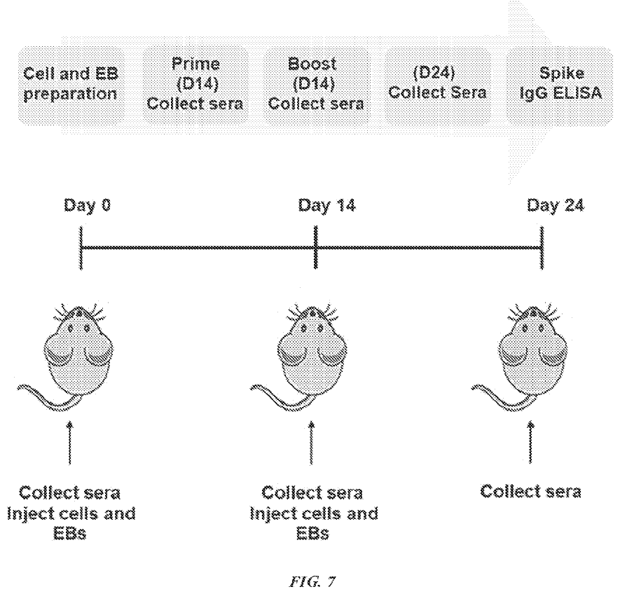

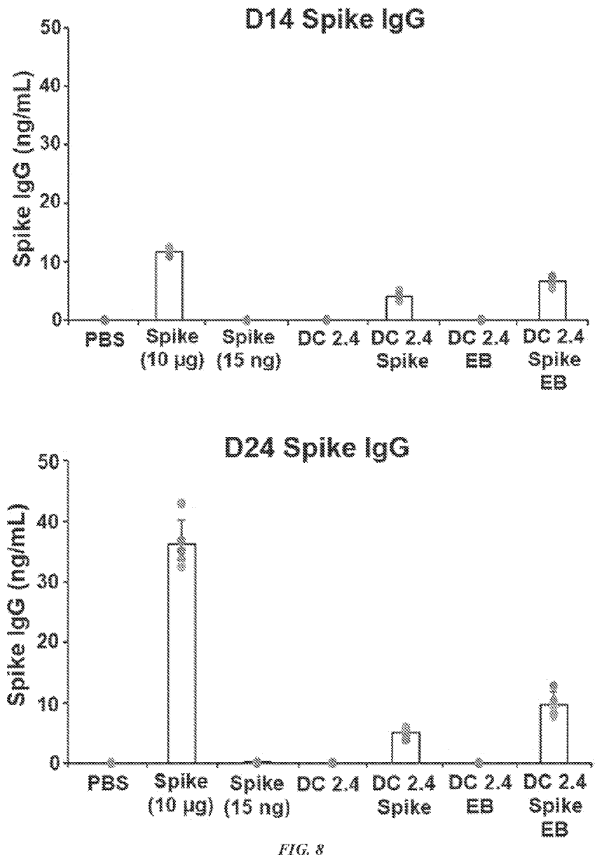

Scheme of in vivo vaccination. C57BL/6 mice were immunized twice (prime and booster) via subcutaneous injection 14 days apart. Sera was collected at the time of the prime shot (Day 0) and booster shot (Day 14) as well as 10 days after the booster shot (Day 24) (see FIG. 7).

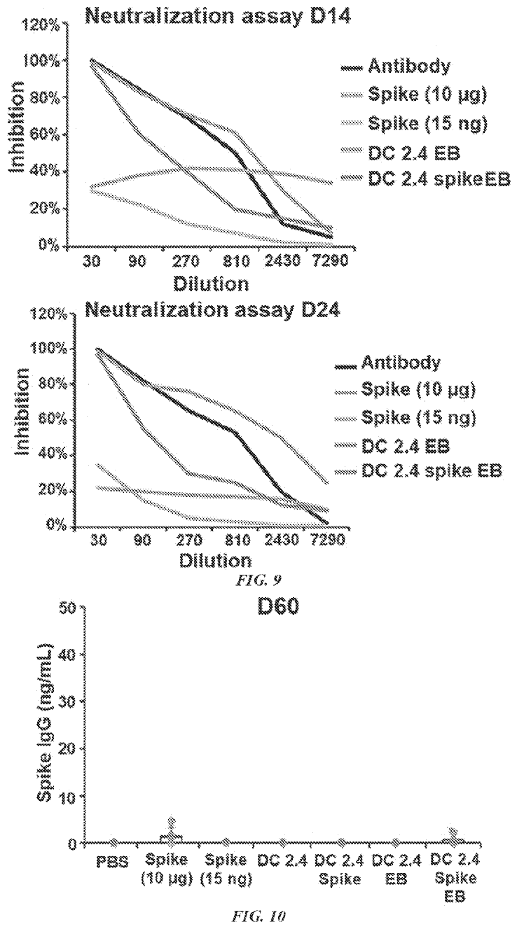

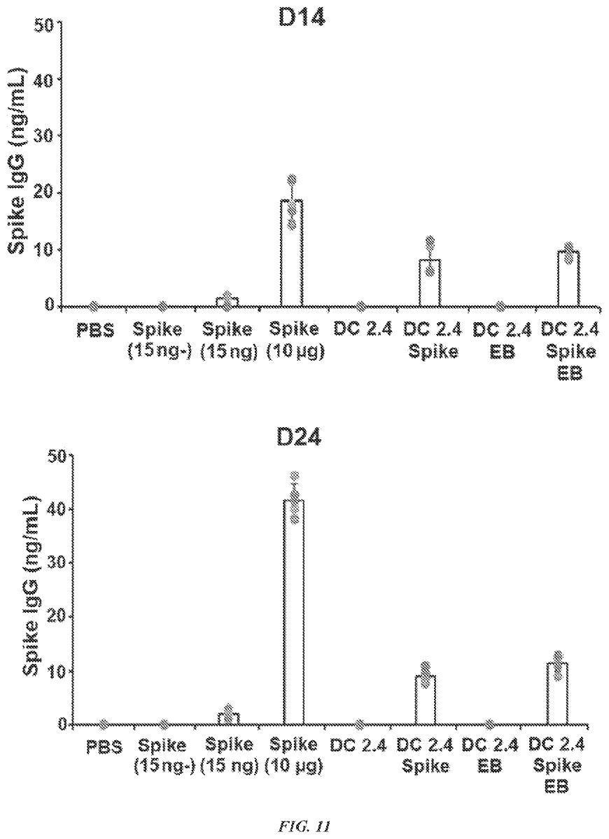

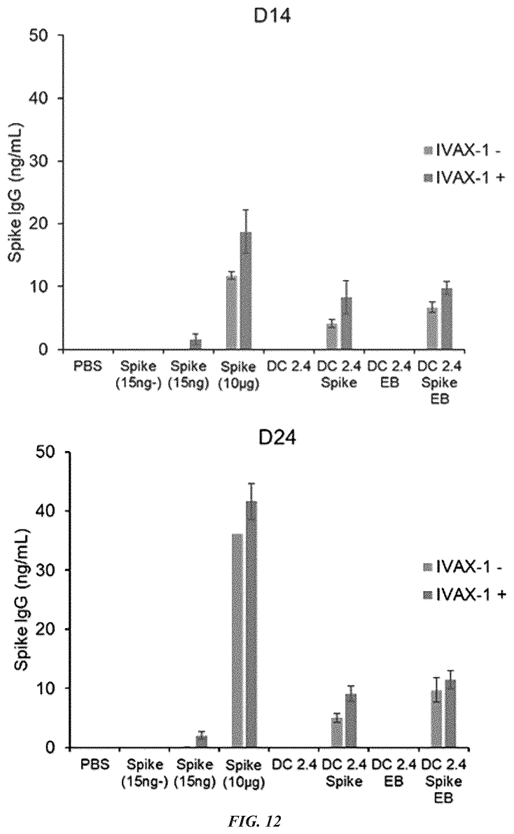

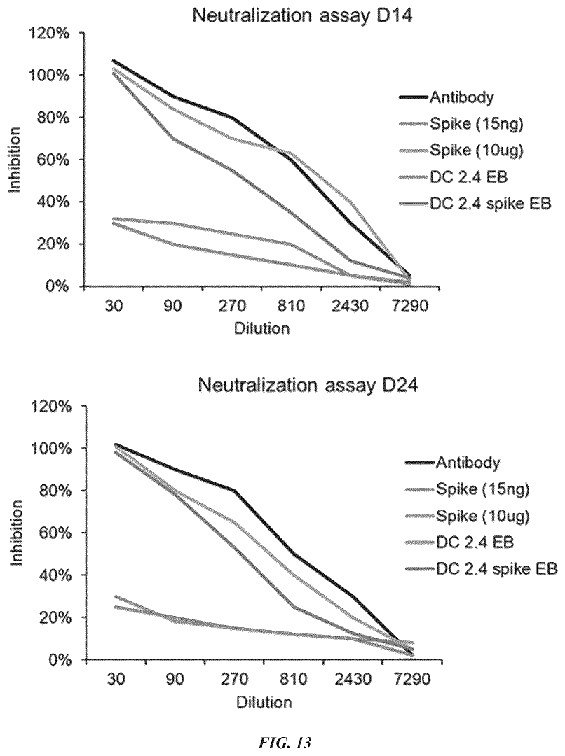

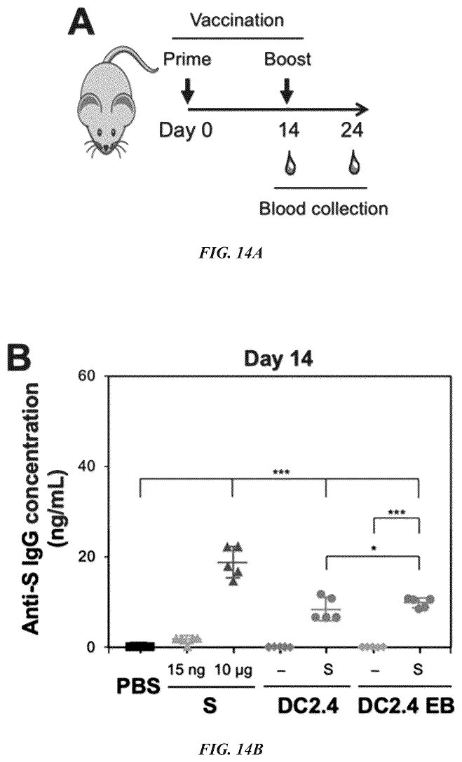

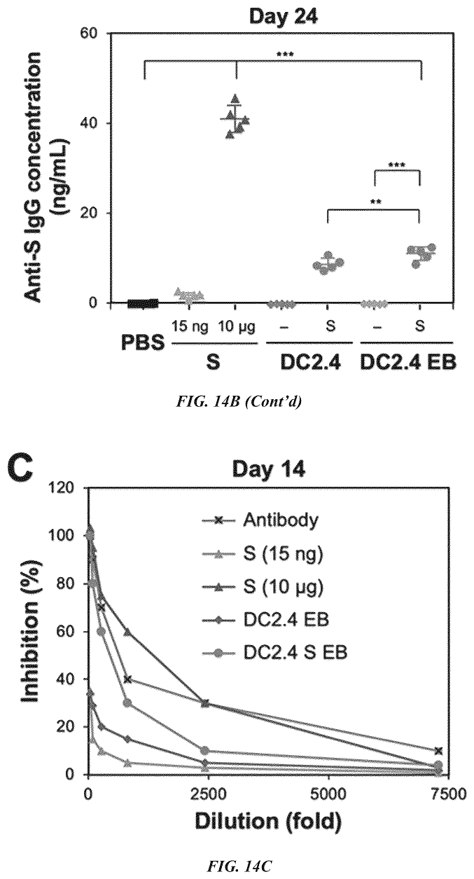

Amount of spike expression in DC 2.4 spike cells and DC 2.4 spike EBs. Prior to immunization of C57BL/6 mice with 2.5×10⁵ DC 2.4 and DC 2.4 spike cells, DC 2.4 spike cells and EBs were analyzed using an ELISA to determine the amount of spike expression in 2.5×10⁵ cells and equivalent amount by surface area of 2.5×10⁵ DC 2.4 spike EBs (see FIG. 8). DC 2.4 spike cells and EBs were collected and lysed using 1× RIPA buffer and analyzed for spike expression. Pre-coated spike ELISA wells were washed with washing buffer and 100 μL of cell lysate samples were added to the wells and incubated for 2 hours at room temperature. Detection antibody against spike antibody (HRP-linked 2° Ab) was added to each well and absorbance was measured at 450 nm. In a total of 300 μL. there was approximately 15 ng of spike protein present and for further immunization studies, an equivalent spike dose (15 ng) to 2.5×10⁵ DC 2.4 spike cells were used.

Anti-spike protein IgGs from mice vaccinated subcutaneously with free spike protein, DC 2.4 cells. DC 2.4 spike cells and their corresponding extracellular blebs. C57BL/6 mice were vaccinated twice with free spike protein (10 μg/mouse), equivalent amount of spike protein for 2.5×10⁵ cells (15 ng/mouse), 2.5×10⁵ DC 2.4 and DC 2.4 spike cells, an equivalent amount by surface area of 2.5×10⁵ DC 2.4 and DC 2.4 spike EBs via subcutaneous injection 14 days apart. Sera was collected at the time of the prime shot (Day 0) and booster shot (Day 14) as well as 10 days after the booster shot (Day 24), the collected sera was centrifuged at 6000×g for 10 minutes and stored in −80° C. to maintain its stability. Sera spike IgG was detected using ELISA. Recombinant SARS-CoV-2 spike protein was coated at 2 μg per well overnight. Blocking buffer was added to each well and incubated at room temperature for 2 hours. The serum samples were diluted by 20-fold and added to the wells and incubated for 2 hours at RT. Detection antibody (mouse IgG-HRP) was added to each well and incubated for 1 hour at RT. TMB substrate was added to each well and the reaction was stopped using stop solution and absorbance was detected at 450 nm and 570 nm The results showed efficient antibody generation by spike expressing EBs against the SARS-CoV-2 S protein, while no activation by control DC 2.4 EB was observed. Importantly, DC 2.4 spike EBs were more efficient than spike expressing dendritic cells by 1.4-2.0-fold and free S protein ˜335-fold in spike antibody generation.

Neutralization assay from sera obtained from vaccinated mice. Viruses such as the coronavirus SARS-CoV-2 (2019-nCoV), SARS-CoV, Ebola virus, H5N1, etc., are highly infectious and highly pathogenic, which have brought great difficulty and danger to the screening of neutralizing antibodies. Compared with a natural virus. the pseudovirus can only infect cells in a single round. has broad host range, high titer, and is not easily inactivated by serum complement. Sera were collected from immunized mice with spike protein (10 μg/mouse), equivalent amount of spike protein for 2.5×10⁵ cells (15 ng/mouse), equivalent amount by surface area of 2.5×10⁵ DC 2.4 and DC 2.4 spike EBs. The mice were immunized twice 14 days apart (prime and booster), and sera was collected at the time of prime, booster and 10 days after booster shot. As a positive control, monoclonal antibody against SARS-CoV-2 spike protein was used to monitor variability of the neutralization experiments. The sera samples from the spike protein showed neutralizing activity against SARS-CoV-2 spike pseudotyped viruses with neutralizing titers ranging from 1:30 to 1:270. The sera samples from DC 2.4 spike EB showed neutralizing activity at 1:30 while the serum samples obtained from DC 2.4 EB. and spike protein (15 ng/mouse) did not show any effective neutralizing activity.

Long-term effect of anti-spike protein IgGs from mice vaccinated subcutaneously with free spike protein, DC 2.4 cells. DC 2.4 spike cells and their corresponding extracellular blebs. C57BL/6 mice vaccinated twice with free spike protein (10 μg/mouse). equivalent amount of spike protein for 2.5×10⁵ cells (15 ng/mouse), 2.5×10⁵ DC 2.4 and DC 2.4 spike cells, an equivalent amount by surface area of 2.5×10⁵ DC 2.4 and DC 2.4 spike EBs via subcutaneous injection 14 days apart. Sera was collected at D60 after prime shot, the collected sera were centrifuged at 6000×g for 10 minutes and stored in −80° C. to maintain its stability. Sera spike IgG was detected using ELISA. Recombinant SARS-CoV-2 spike protein was coated at 2 μg per well overnight. Blocking buffer was added to each well and incubated at room temperature for 2 hours. The serum samples were diluted by 20-fold and added to the wells and incubated for 2 hours at RT. Detection antibody (mouse IgG-HRP) was added to each well and incubated for 1 hour at RT. TMB substrate was added to each and the reaction was stopped using stop solution and absorbance was detected at 450 nm and 570 nm. The results showed a decrease of antibody generation in the spike (10 μg), and spike expressing EBs against the SARS-CoV-2 S protein.

Anti-spike protein IgGs from mice vaccinated subcutaneously with adjuvanted spike protein. DC 2.4 cells, DC 2.4 spike cells and their corresponding extracellular blebs. C57BL/6 mice were vaccinated twice with adjuvanted spike protein (10 μg/mouse). equivalent amount of spike protein for 2.5×10⁵ cells (15 ng/mouse), 2.5×10⁵ DC 2.4 and DC 2.4 spike cells, an equivalent amount by surface area of 2.5×10⁵ DC 2.4 and DC 2.4 spike EBs via subcutaneous injection 14 days apart. Sera was collected at the time of the prime shot (Day 0) and booster shot (Day 14) as well as 10 days after the booster shot (Day 24). The collected sera were centrifuged at 6000×g for 10 minutes and stored in −80° C. to maintain its stability. Adjuvants are used to augment and orchestrate immune responses and influence the functional profile of B and T cell responses. Cationic adjuvants have been shown to induce strong T cell responses when tested with protein-based antigens. Addition of cationic liposome adjuvant (IVAX-1) showed efficient antibody generation by spike expressing EBs against the SARS-CoV-2 S protein, while no activation by control DC 2.4 EB was observed. However, as compared to mice immunized without adjuvants, there was not a big increase in spike antibody generation.

Neutralization assay from sera obtained from adjuvant vaccinated mice. Sera were collected from immunized mice with spike protein (10 μg/mouse), equivalent amount of spike protein for 2.5×10⁵ cells (15 ng/mouse), equivalent amount by surface area of 2.5×10⁵ DC 2.4 and DC 2.4 spike EBs in presence of an IVAX-1 adjuvant. The mice were immunized twice 14 days apart (prime and booster), and sera was collected at the time of prime, booster and 10 days after booster shot. As a positive control, monoclonal antibody for SARS-CoV-2 spike protein was used to monitor variability of the neutralization experiments. The sera samples from the spike protein showed neutralizing activity against SARS-CoV-2 spike pseudotyped viruses with neutralizing titers that ranged from 1:30 to 1:810. The sera samples from DC 2.4 spike EB showed neutralizing activity at 1:30. While the serum samples obtained from DC 2.4 EB, and spike protein (15 ng/mouse) did not show any neutralizing activity (see FIG. 16).

To investigate the DC 2.4 S EBs ability to induce neutralizing antibodies against SARS-CoV-2 spike-expressing virus, C57BL/6 mice were vaccinated twice with PBS, S (10 μg as a conventional vaccination dose), S (15 ng; equivalent to the S amount in 2.5×10⁵ DC2.4 S and DC2.4 S EBs), 2.5×10⁵ DC2.4 cells or DC2.4 S cells, and DC2.4 EBs or DC2.4 S EBs an equivalent surface area of 2.5×10⁵ of the corresponding cells. The EB's were subcutaneously injected 14 days apart at an equivalent amount of the corresponding cells by surface area. Plasma for anti-S IgG and virus neutralization assays were collected at the time of the prime and booster as well as 10 days after the booster (see FIG. 14A) DC2.4 S and DC2.4 S EBs generated similar levels of anti-S antibodies, while no antibody production was observed by S-free DC2.4 cells and their EBs (see FIG. 14B). Notably, the antibody production by DC2.4 S EBs was slightly higher than that of DC2.4 S cells, especially 10 days after the booster, indicating efficient activation of humoral immunity by DC2.4 S EBs in vivo. Compared to an equivalent amount of vaccinated S (15 ng per mouse), DC2.4 S EBs were substantially more efficient (˜350 fold) than the free S in driving antibody production. The booster increased IgG production by ˜2-fold with S (10 μg per mouse) but not IgG production by DC2.4 S EBs, implying the feasibility of achieving single-shot vaccination. The use of an adjuvant, IVAX-1 increased antibody production by S vaccines (15 ng per mouse) by 5-fold (see FIG. 15). In contrast, IVAX-1 marginally affected the antibody production by DC2.4 S and DC2.4 S EBs, likely because these vaccines are already equipped for T cell activation.

Virus neutralization using a pseudotyped lentivirus was observed with the plasma samples collected from the mice vaccinated with S (10 μg per mouse) and DC2.4 S EBs (see FIG. 14C). In contrast to a wild type SARS-CoV-2, a pseudotyped lentivirus can only infect cells in a single round, has broad range of host cells, prepared at a high titer, and is not easily inactivated by serum complement. Despite the ˜670-fold difference in dose of S, the animals vaccinated with 10 μg had similar, though slightly higher neutralizing antibody responses as the animals vaccinated with DC2.4 S EBs, indicating the high neutralizing efficacy by DC2.4 S EBs. Vaccination with either DC2.4 EBs or S at 15 ng resulted in little if any neutralizing antibody response. These results demonstrate that S-presenting DC-derived EBs given at a very low dose were efficient in producing anti-S antibody that is capable of neutralizing S-pseudotyped virus.

It will be understood that various modifications may be made without departing from the spirit and scope of this disclosure. Accordingly, other embodiments are within the scope of the following claims.

1. A vaccine preparation comprising extracellular blebs from a cell that has been genetically engineered to express an antigen(s),
wherein the extracellular blebs are produced from the cell by treating the cell with a blebbing agent, and
wherein the antigen is displayed on the surface of the extracellular blebs.

2. The vaccine preparation of claim 1, wherein the cell is selected from a macrophage, a B cell and a dendritic cell.

3. (canceled)

4. The vaccine preparation of claim 1, wherein the cell is an immortalized antigen presenting cell.

5. The vaccine preparation of claim 1, wherein the cell is a human primary cell, or
the cell is differentiated from human embryonic stem cells (hESCs) or induced pluripotent stem cells (iPSCs) from a human subject.

6. (canceled)

7. The vaccine preparation of claim 1, wherein the antigen(s) is a foreign antigen(s) or an endogenous antigen that has been genetically modified for improved therapeutic outcomes.

8. The vaccine preparation of claim 7, wherein the foreign antigen(s) is from a pathogenic or disease-causing microorganism selected from a bacterium, a fungus, and a virus.

9-13. (canceled)

14. The vaccine preparation of claim 1, wherein the antigen is a cancer or tumor antigen.

15. The vaccine preparation of claim 1, wherein a viral vector is used to genetically engineer the antigen presenting cell to expresses the antigen(s).

16. The vaccine preparation of claim 15, wherein the viral vector is a lentivirus vector, an adenovirus vector, an adeno-associated virus vector, or a gammaretrovirus vector.

17. (canceled)

18. The vaccine preparation of claim 1, wherein the vaccine preparation further comprises an adjuvant.

19. (canceled)

20. The vaccine preparation of claim 1, wherein the extracellular blebs comprise one or more of the following surface and maturation markers CD11c, MHC I, CD40, CD80, and/or CD86.

21. A method of making a vaccine preparation of claim 1, comprising:
generating extracellular blebs from a genetically engineered cell by contacting the cell with the one or more sulfhydryl blocking agents for 3 min to 24 h;
isolating the extracellular blebs.

22. (canceled)

23. The method of claim 21, wherein the one or more sulfhydryl blocking agents is N-ethylmaleimide.

24. (canceled)

25. The method of claim 21, wherein the cell is selected from a macrophage, a B cell and a dendritic cell.

26. (canceled)

27. The method of claim 21, wherein the cell is an immortalized antigen presenting cell.

28. The method of claim 21, wherein the cell is a human primary cell, or
the cell is differentiated from human embryonic stem cells (hESCs) or induced pluripotent stem cells (iPSCs) from a human subject.

29. (canceled)

30. The method of claim 21, wherein the antigen(s) is a foreign antigen(s) from a pathogenic or disease-causing microorganism selected from a bacterium, a fungus, and a virus.

31-36. (canceled)

37. The method of claim 21, wherein the antigen is a cancer or tumor antigen.

38. The method of claim 21, wherein the genetically engineered cells are made by transforming the cells with a viral vector that encodes the antigen(s)

39-40. (canceled)

41. A method of immunizing a subject, comprising administering a therapeutically effective amount of the vaccine preparation of claim 1 to the subject.
